(19) United States
(12) Patent Application Publication
(10) Pub. No.: US 2025/0295341 A1
(43) Pub. Date: Sep. 25, 2025

(54) HELMET SYSTEM AND KIT

(71) Applicant: VIRGINIA TECH INTELLECTUAL PROPERTIES, INC., Blacksburg, VA (US)

(72) Inventors: Brittany Howell, Roanoke, VA (US); Megan Evans, Roanoke, VA (US); Claudia Carreno, Roanoke, VA (US); Jamie Holt, Roanoke, VA (US); Saket Bikmal, Roanoke, VA (US); Mateo McDermott, Blacksburg, VA (US)

(21) Appl. No.: 19/086,230

(22) Filed: Mar. 21, 2025

Related U.S. Application Data
(60) Provisional application No. 63/568,068, filed on Mar. 21, 2024.
(60) Provisional application No. 63/661,938, filed on Jun. 20, 2024.

(51) Int. Cl. A61B 5/245; A42B 3/04; A61B 5/00

(52) U.S. Cl. CPC A61B 5/245; A42B 3/0433; A61B 5/6803

(57) ABSTRACT

The present disclosure provides for a helmet system for use in MEG scans of a pediatric subject, such as a human subject, and methods of making the helmet system. MEG is a noninvasive imaging technique for capturing brain activity by measuring small magnetic fields produced in the brain, where the helmet system can be used for MEG scans to detect, for example, sleep disturbances, seizures, and motor disorders.

### CROSS-REFERENCE TO RELATED APPLICATIONS

This application claims the benefit of: U.S. Provisional Patent Application No. 63/661,938 filed on Jun. 20, 2024 and U.S. Provisional Patent Application No. 63/568,068 filed on Mar. 21, 2024, each of which is incorporated herein by reference in its entirety.

### STATEMENT ON FUNDING PROVIDED BY THE U.S. GOVERNMENT

This invention was made with Government support under grant number R21EB031547 awarded by the National Institute of Biomedical Imaging and Bioengineering. The Government has certain rights in the invention.

### BACKGROUND

Social and physical interactions with other humans have a fundamental role in determining human brain function and health across the lifespan. Yet, to date, human brain imaging systems have struggled in their ability to noninvasively measure functional brain activity in naturalistic contexts with complex movements, such as face-to-face social interactions or tasks requiring whole-body motion, which is particularly true during the first years of life.

### SUMMARY

The present disclosure provides for a helmet system for use in MEG scans of a pediatric subject, such as a human subject, and methods of making the helmet system.

The present disclosure provides for a helmet system, comprising: a helmet shell configured to fit the head of a human subject, the helmet shell weighing approximately 450 grams or less; at least one sensor housing unit, configured to hold a magnetoencephalography field sensor; and a plurality of openings disposed in the helmet shell, wherein at least two individual openings of the plurality of openings are configured to receive and securely fit the sensor housing unit.

The present disclosure provides for a kit, comprising: the helmet system as described above and herein; and at least one magnetoencephalography field sensor

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates a schematic overview of an OPM-MEG system.

FIG. 2A shows a MSR schematic.

FIG. 2B shows nulling coils of an MSR.

FIG. 3A illustrates an OPM-MEG design layout of the interior of an MSR; panel adapted from Holmes et al., 2023a.

FIG. 3B shows a photograph of an interior of an MSR.

FIG. 3C shows a time series of MSR field changes measured by 12 dual-axis (Y and Z) OPM sensors (QuSpin, gen-2 QZFM) during a 20-minute empty-room recording with nulling off for the first 10 minutes followed by 10 minutes with active nulling on. Insert: maximum field change across sensors with nulling off (blue bar; mean (std)=0.95 nT (0.57)) and on (red bar; mean (std)=0.25 nT (0.12)).

FIG. 4A shows adult OPM-MEG source imaging co-registration of OPM sensors to individual brain space. Top: location of a whole-head OPM sensor array using a one-size-fits-most rigid helmet (Cerca Magnetics large adult helmet); insert: alignment of helmet (purple) with individual structural MRI (grey) via 3D model of participant wearing helmet (green).

FIGS. 4B-4C show a source-localization beamformer analysis (top) and virtual electrode time series reconstruction and spectrogram (bottom) from adult OPM-MEG source imaging of (FIG. 4B) visual-evoked gamma oscillations during a visual-cued finger tapping task and (FIG. 4C) finger movement-evoked beta band desynchronization response.

FIG. 4D beta band beamformer results of adult OPM-MEG source imaging from individual native space in FIG. 4C normalized to Montreal Neurological Institute template brain space.

FIG. 5 shows a plot of the power spectral density of sensor-level data recorded from an adult performing an instructed resting state task alone (blue) and with their child (red) and from an adult during a parent-infant interaction task (magenta). 12 dual-axis OPMs placed around the head were used for these recordings. Insert: power spectral density on 0-15 Hz to highlight low-frequency power related to differing levels of motion across tasks.

FIG. 6A shows an outline of Experiment 2's 2×2 design comparing combinations of sensor mounting strategy (rigid helmet, flexible cap) and source model (native brain, template brain).

FIG. 6B shows a photograph of an infant wearing a flexible cap with OPM sensors; adapted from Feys et al., 2023.

FIG. 6C shows 3D-printed OPM sensor holders used at to mount sensors onto flexible caps.

FIG. 6D shows a rigid helmet for infant OPM-MEG recordings. The yellow circle shows position of sensor data shown in FIG. 9C.

FIG. 7A shows a power spectral density plot of six infant OPM-MEG recordings across three infants wearing rigid caps.

FIG. 7B shows a raw sensor-level time series (from black power spectral density trace in FIG. 7A), showing rail-to-rail limits (bold horizontal lines) and saturation of OPM sensor field measurements. Nine sensors are plotted; the sensor's Y-axis data is in red and Z-axis in blue.

FIG. 8 shows examples of infant T1 and T2 weighted MRI.

FIGS. 9A-9D show representative hyperscanning of source activity during parent-infant interactions. For this setup, a mother-infant dyad was seated in the center of an MSR with six sensors placed over the left sensorimotor cortex of the infant using a rigid helmet and six for the parent. FIG. 9A shows power spectral density of sensor-level data from the infant (blue) and the parent (red) during the visuomotor task. FIG. 9B shows a trial-averaged sensor level data for a sensor on the infant scalp (highlighted in FIG. 6D). There were 43 useable trials of infant OPM data. FIG. 9C shows sensor co-registration to template adult MRI and source reconstruction of beta band activity showing localization to left sensorimotor cortex. SPMs show top 5% of voxels. FIG. 9D shows trial-averaged time series of source-adult beta band activity and infant sensor-level beta band activity during visuomotor task.

FIG. 10 shows CAD imaged of an OPM-MEG helmet design side view (a) top view (b) and front view (c)

FIG. 11 shows an OPM-MEG cap adaptation for supine positioning.

FIGS. 12A-12C shows an OPM-MEG cap customization, including a side view of a soft inner cap (FIG. 12A), a front view of a soft inner cap (FIG. 12B), and a representative cap design with mounted sensors (FIG. 12C).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of material chemistry, biomedical engineering, and the like, which are within the skill of the art. Such techniques are explained fully in the literature.

The following examples are put forth so as to provide those of ordinary skill in the art with a complete disclosure and description of how to perform the methods and use the devices and systems disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 weight percent to about 5 weight percent but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range. The term “about” can include traditional rounding according to significant figures of the numerical value. In addition, the phrase “about ‘x’ to ‘y’” includes “about ‘x’ to about ‘y’”.

Furthermore, the terms “about”, “approximately”, “at or about”, and “substantially” as used herein mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated+10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “an opening” includes a plurality of openings. In this specification and in the claims that follow, reference will be made to a number of terms that shall be defined to have the following meanings unless a contrary intention is apparent.

### Abbreviations

EEG—electroencephalography
MEG—magnetoencephalography
MRI—magnetic resonance imaging
MSR—magnetically shielded room
OFC—occipital frontal circumference
OPM—optically pumped magnetometer
SQUID—superconducting quantum interference device

### DISCUSSION

The present disclosure provides for a helmet system for use in MEG scans of a pediatric subject, such as a human subject, and methods of making the helmet system. MEG is a noninvasive imaging technique for capturing brain activity by measuring small magnetic fields produced in the brain. The helmet system can be used for MEG scans to detect, for example, sleep disturbances, seizures, and motion disorders. The system can further provide early identification and low-risk monitoring of neurological conditions in a subject.

There are limitations in current brain activity screening tools for babies, including EEG and functional magnetic resonance imaging. This is evident in the inferior spatial resolution of source localization of brain activity (EEG) and a subject's sound discomfort and movement restriction (MRI), which may not give appropriate representations of infant brain activity for effective screening. Current OPM-MEG helmet systems or caps are configured for use by adults. However, the size, weight, and stiffness of these existing caps do not accommodate the needs of infants. Due to variability in infant head shape and size, merely downsizing existing adult helmet systems leaves substantial gaps between the helmet system or cap and the skull of an infant subject. This can reduce the spatial resolution of the OPMs and result in erroneous sensor readings. Additionally, even with downsizing, existing helmet systems are too heavy for use by an infant. The helmet system of the present disclosure is light in weight, able to effectively dissipate heat, and, optionally, is customizable to a subject's head. The helmet system is also sturdy enough to allow smooth flexible movements and comfort for a subject for accurate readings. In addition, the device allows for real-time study of a subject's brain signals while freely moving during various physical activities, which can provide valuable information in neurodevelopmental research and for studies that track the onset of seizures in high-risk individuals, such as infants.

### Helmet System and Kit

Disclosed herein is a helmet system including: a helmet shell configured to fit the head of a human subject, where the helmet shell weighs approximately 450 grams or less; at least one sensor housing unit, configured to hold a MEG field sensor (e.g., an OPM); and a plurality of openings disposed in the helmet shell, where at least two individual openings of the plurality of openings are configured to receive and securely hold a single sensor housing unit. The helmet system can further include at least one inflatable air bladder or other padding attached to an inner surface of the helmet shell. The inflatable air bladder or additional padding can be comprised of a high molecular weight polyethylene, or a derivative thereof or heat tolerant based foam.

Human infants can undergo relatively rapid changes in head size and shape during the first year of life. The average human head circumference or OFC grows by 4 cm from 3 months to 6 months, slowing down to 2 cm between 6 months and 9 months and 1 cm between 9 months and 12 months. During this period, infant head shape is malleable in response to positioning, movement, intracranial content, and fusion of the sutures. The helmet shell can be configured to fit the unique head shapes of infants, including typical head shapes (normocephaly) and atypical head shapes, such as brachycephaly, plagiocephaly, scaphocephaly, trigonocephaly, asymmetrical ears, or a combination thereof.

In one aspect, the helmet system or helmet shell can be configured to fit the head of a human subject of approximately 2 months to approximately 24 months old, approximately 2 months to approximately 22 months old, approximately 2 months to approximately 20 months old, or approximately 2 months to approximately 18 months old. In another aspect, the helmet system or helmet shell can be configured to fit the head of a human subject with an OFC of approximately 35 cm to approximately 55 cm, approximately 35 cm to approximately 50 cm, approximately 35 cm to approximately 45 cm, or approximately 35 cm to approximately 40 cm. In another aspect, the helmet system or helmet shell can be configured to fit the head of a human subject with an OFC of approximately 35 cm to no greater than 53 cm, approximately 35 cm to no greater than 51 cm, approximately 35 cm to no greater than 49 cm, approximately 35 cm to no greater than 47 cm, or approximately 30 cm to no greater than 53 cm, approximately.

Also disclosed herein is a kit including any configuration of the helmet system disclosed herein and at least one MEG field sensor. The kit can further include a cap configured to fit the head of a human subject. MEG field sensors can heat up during scanning, and a barrier between the head of a subject and the sensors can reduce any discomfort a subject may experience. The cap can provide a barrier between the helmet system and the head of a subject. In another aspect, the cap can be comprised of a material with good thermal insulation, such as cotton, neoprene, thermoplastic polyurethane (TPU), nylon coated with TPU, polyester, or a combination thereof. In another aspect, the cap can be comprised of a material with good thermal conductivity, such as high molecular weight polyethylene. In one aspect, the cap can be configured to securely attach to the helmet shell, such as via a hook and loop fastener. In another aspect, the cap can be configured to be securely attached to a subject's head, such as by a chin strap that is secured by tying, by a hook and loop fastener, by a buckle, or the like.

In one aspect, the helmet shell can be configured to extend from the frontal region of a subject's head, e.g., from just above the supraorbital ridges, to the parietal region or occipital region of a subject's head. The helmet shell can extend from the frontal region to cover all or most of the parietal region and all or most of the occipital region. In another aspect, the helmet shell can be configured to only cover part of the frontal region, the parietal region, and/or the occipital region of a subject's head. In another aspect, the helmet shell can be configured to leave all or part of the frontal region, the parietal region, and/or the occipital region of a subject's head uncovered. For example, the helmet shell can be configured to cover the front region of a subject's head, e.g., starting at or extending from just above the supraorbital ridges, and leaving at least the occipital region of a subject's head uncovered or all or most of the parietal region of a subject's head uncovered. In a further aspect, the helmet shell can be configured to extend from the front of a subject's head, just above the supraorbital ridges, to the vertex of a subject's head, covering all or part of the frontal region of a subject's head and leaving at least the occipital region of a subject's head uncovered by the helmet shell when worn. The vertex of a human subject's head, as used herein, refers to the highest point of the skull, located near the midpoint of the sagittal suture. In one aspect, the helmet shell can be configured to not cover a subject's ears when worn.

The helmet shell can have a thickness ranging from approximately 1 mm to approximately 6 mm or approximately 2 mm to approximately 5 mm. The longest length between the front of the helmet shell (e.g., the portion of the helmet shell configured to be in contact with the frontal region of a subject's head) and the back of the helmet shell (e.g., the portion of the helmet shell configured to be in contact with the occipital region or the parietal region of a subject's head) can range from approximately 100 mm to approximately 170 mm, approximately 110 mm to approximately 160 mm, approximately 100 mm to approximately 120 mm, approximately 110 mm to approximately 120 mm, approximately 150 mm to approximately 170 mm, or approximately 150 mm to approximately 160 mm. The longest width between two sides of the helmet shell (e.g., the portions of the helmet shell configured to be in contact with or near to the temporal region of a subject's head) can range from approximately 70 mm to approximately 140 mm, approximately 80 mm to approximately 140 mm, approximately 90 mm to approximately 130 mm, approximately 70 mm to approximately 90 mm, approximately 80 mm to approximately 90 mm, approximately 120 mm to approximately 140 mm, or approximately 120 mm to approximately 130 mm.

The openings disposed in the helmet shell can individually be circular, oval, or polygonal in shape, where the longest diameter of each opening (e.g., the major axis of an oval) ranges from approximately 5 mm to approximately 25 mm, approximately 5 mm to approximately 20 mm, approximately 10 mm to approximately 25 mm, approximately 10 mm to approximately 20 mm, or approximately 15 mm to approximately 25 mm.

The helmet shell can weigh from approximately 100 grams to approximately 450 grams, approximately 100 grams to approximately 400 grams, approximately 100 grams to approximately 350 grams, approximately 100 grams to approximately 300 grams, approximately 150 grams to approximately 400 grams, approximately 150 grams to approximately 350 grams, approximately 150 grams to approximately 300 grams, approximately 150 grams to approximately 250 grams, or approximately 175 grams to approximately 225 grams. In one aspect, the light weight of the helmet shell can make the system appropriate for use by a subject that cannot support a heavier helmet, such as an infant. In one aspect, the helmet shell can be comprised of a relatively rigid material. The helmet shell can be comprised of polylactic acid, polyethyleneterephthalate glycol, or a combination thereof. The helmet shell can be configured to be securely attached to a subject's head, such as by a chin strap that is secured by tying, by a hook and loop fastener, by a buckle, or the like.

The helmet system (including helmet shell and sensor housing unit(s)) can weigh from approximately 100 grams to approximately 450 grams, approximately 100 grams to approximately 400 grams, approximately 100 grams to approximately 350 grams, approximately 100 grams to approximately 300 grams, approximately 150 grams to approximately 450 grams, approximately 150 grams to approximately 400 grams, approximately 150 grams to approximately 350 grams, approximately 200 grams to approximately 450 grams, approximately 200 grams to approximately 400 grams, or approximately 200 grams to approximately 350 grams.

The helmet system can further include at least one sensor housing unit configured to hold a MEG field sensor. In one aspect, the housing unit can comprise a semi-flexible material that allows for the housing unit to be inserted into or removed from any one of the openings in the helmet shell configured to receive a housing unit. The housing unit can be securely held or fixed within an opening in the helmet shell using a snap-fit, screws, or rivets. The housing unit can be comprised of thermoplastic copolyester, thermoplastic polyurethane, or a combination thereof.

Once a sensor housing unit has been inserted into an opening in the helmet, the unit can be rotated a full 360 degrees prior to or after being fixed within the opening. The sensor housing units are configured so that, when a MEG field sensor is placed within a housing unit, the sensor can be moved within the housing unit towards or away from a subject's head. In one aspect, the sensor housing unit is configured to at least partially cover a MEG field sensor that is placed within the unit, and moving the sensor within the housing unit towards or away from a subject's head allows for more or less of the sensor to be covered by the housing unit. The depth at which a sensor sits in a housing unit, measured as the distance from the bottom of the sensor to the top of the housing unit, can be adjusted to allow for more or less coverage of the sensor. For example, a sensor can sit in a housing unit at a depth of approximately 19 mm (e.g., 19.4 mm). In one aspect, the housing unit can be configured so that it covers approximately half of a sensor when the sensor sits in the housing unit at a depth of approximately 19 mm. The depth can be adjusted to improve heat dissipation or more securely hold the sensor in place.

The MEG field sensor of the kit can be an OPM sensor. In one aspect, the OPM sensor can be a diaxial sensor configured to measure two spatial component vectors of a magnetic field relative to a subject's head. Recent advancements have allowed for the creation of triaxial sensors configured to measure three spatial component vectors. Both diaxial and triaxial sensors are compatible with the helmet system.

In one aspect, the plurality of openings can comprise at least two openings configured to receive and securely hold a single sensor housing unit. In a further aspect, each opening in the helmet shell can be configured to receive and securely hold a single sensor housing unit. In another aspect, the helmet system can include from 2 to approximately 50 openings in the helmet shell. At least one of the plurality of openings configured to hold a sensor housing unit can be left open (i.e., without a sensor housing unit in place) to allow for improved heat dissipation from the helmet system.

### Method of Making and of Use of the Helmet System and Kit

In one aspect, the helmet shell described herein can be fabricated in a variety of standardized sizes, such as standardized sizes for infants. These standardized sizes can be fabricated based on an average head circumference or OFC of a subject at different stages. OFC measurements can be obtained by measuring the widest circumference of a subject's head, between the most prominent part of the occiput and just above the supraorbital ridges. of the distance around of back of the head of a subject. In one aspect, standard helmet shell sizes can be configured to fit subjects with an OFC ranging from approximately 24 cm to approximately 51 cm, approximately 24 cm to approximately 49 cm, or approximately 26 cm to approximately 51 cm. The standard helmet shell sizes can be configured so that they will fit subjects whose heads fall within a given range of OFC size. For example, standard helmet shell sizes can be configured to fit a subject with up to a 2 cm variation in OFC. As a further example, a standard helmet shell size configured to fit a subject with an OFC of approximately 31 cm to approximately 33 cm, approximately 32 cm to approximately 34 cm, or approximately 33 cm to approximately 35 cm could fit a subject with an OFC of approximately 33 cm. In another aspect, a standard helmet shell size can be configured to fit a subject with up to a 1 cm variation in OFC or up to a 3 cm variation in OFC.

In another aspect, the helmet shells described herein can be fabricated based on an individual subject's head shape and size. A reference picture or measurements can be taken of a subject's head and used to guide helmet design and fitting. Reference pictures can include a side view, front view, or back view of a subject's head. In one aspect, the measurements used to guide fabrication of an individual subject's helmet include the subject's OFC and/or the distance from the inion to the nasion of the subject's head.

The helmet shells and sensor housing units can be fabricated by 3D printing, casting, injection moulding, or the like.

In one aspect, the helmet kit can be used to take MEG scans using the following procedure. At least two sensor housing units comprising MEG sensors and, optionally, additional empty sensor housing units can be placed into openings in the helmet shell. The helmet system can then be placed onto a subject's head and secured. Optionally, a cap can be attached to the helmet shell or secured to a subject's head prior to placing and securing the helmet system on the subject. MEG scans can be performed while the subject is in a magnetically shielded room that has been degaussed. Additional persons, such as a caregiver for an infant subject, can also be present in the room. In one aspect, MEG scanning sessions last for no longer than approximately one hour, no longer than approximately 40 minutes, or no longer than approximately 20 minutes. A subject can be placed between nulling coils to diminish impact of ambient magnetic fields while allowing for relatively free movement of the subject.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Example 1

Social and physical interactions with other humans have a fundamental role in determining human brain function and health across the lifespan. Yet, to date, human brain imaging systems have struggled in their ability to noninvasively measure functional brain activity in naturalistic contexts with complex movements, such as face-to-face social interactions or tasks requiring whole-body motion. As a result, understanding of the neural basis of human social and cognitive function during naturalistic social behaviors is greatly lacking. This is particularly true concerning the first years of life, when the foundations of later brain function are being laid. Space constraints and movement-induced signal distortions limit the utility of functional magnetic resonance imaging (fMRI) and traditional MEG systems for social, interactive paradigms involving infants and young children. Ultimately, there remains a need for motion-tolerant neuroimaging technology suitable for all ages and complex multi-person interactions. The lack of such technology is a major barrier to identifying neural markers of social and cognitive functions required for early detection of developmental and neuropsychiatric disorders.

Wearable, room-temperature human MEG systems based on optically-pumped magnetometers (OPMs) holds great promise to become a motion-tolerant and lifespan compliant neuroimaging technology that is suitable for naturalistic social interaction paradigms (Boto et al., 2018; Holmes et al., 2023a). Generic sensor mounting solutions and technology for active magnetic shielding of ambient field fluctuations within magnetically shielded rooms (MSR) have now enabled high-performance OPM-MEG source localization in adults, adolescents, and neonates (Brookes et al., 2022; Hill et al., 2019; Corvilain et al., 2023; Feys et al., 2022). However, there remain inherent limitations of OPM sensor technology, in particular gain errors caused by head motion, which must be addressed to adapt OPM-MEG systems for freely-moving, face-to-face paradigms across all ages. Herein, is discussed an adapted noninvasive neuroimaging method for infants, including recently pioneered OPM-MEG paradigms (Spann et al., 2023; Borna et al., 2020; Feys et al., 2022; Corvilain et al., 2023).

Extant infant OPM-MEG experiments employed knitted, EEG-like caps to mount sensors on the scalp and swaddled infants in a supine position to restrain motion (Feys et al., 2022; Corvilain et al., 2023). Herein, an approach is introduced that used 3D-printed wearable caps for infant OPM-MEG with whole-scalp sensor positioning that are well-tolerated in settings with infants seated upright. The design of OPM arrays uses either rigid helmets or soft caps, and both sensor mounting strategies can be considered for human OPM-MEG studies into the future.

Head movements within the MSR ambient field can distort OPM sensor measurements and impair source reconstruction. Dynamic field correction methods (Robinson et al., 2022; Mellor et al., 2023) can be integrated with head tracking with dynamic control of coils onboard OPM sensors to correct for motion-induced distortions in OPM measurements online during data acquisition.

Being able to assess brain function accurately during natural behaviors, both alone and socially, using OPM-MEG in infancy will provide an opportunity to identify brain signatures of later developing risk. These methods can lay the foundations for currently unattainable goals of early risk identification in naturalistic behavioral and social contexts, and the subsequent early intervention imperative to positive outcomes (Hadders-Algra 2021; Webb et al., 2014). In order to gain the understanding of neurodevelopment needed to accurately identify aberrant patterns of function indicative of risk for developmental and mental health disorders across the lifespan, researchers need to be able to accurately localize and assess brain function during salient natural interactions.

MEG has been applied to understanding infant neurodevelopment using a few behavioral paradigms (Chen et al., 2019) and offers many advantages over more commonly used functional imaging techniques. MEG noninvasively measures human brain electrophysiology via detecting fluctuations in neuromagnetic fields that result from neural communication within the brain. Much like electroencephalography (EEG), MEG provides millisecond temporal resolution; unlike EEG, MEG can be used for precise and accurate signal source localization when combined with high resolution structural imaging (Boto et al., 2018, 2019; Hill et al., 2020), in part because magnetic field fluctuations measured in MEG are not susceptible to distortion or topographical blurring due to differences in conductivity of compartments of brain tissues (e.g. skull, CSF, scalp) as they are for EEG (Hämäläinen et al., 1993). This is particularly important for infant imaging, as the plates that make up the skull are not fused at birth and continue to develop up to 14 months old (Lew et al., 2013). MEG is also quiet and safe, allowing for a relaxed maternal/infant environment, which will likely improve the participant experience, increasing compliance and data retention in the longitudinal applications required to assess developmental trajectories.

Most current MEG systems rely on superconducting quantum interference devices (SQUIDs) that must be cooled using expensive liquid helium to maintain the low temperatures necessary for superconductivity and magnetic flux detection. This results in expensive, large, immobile systems that require participants to remain in a single position. While infant paradigms (Chen et al., 2019) and mother-infant dyadic interactions (Hirata et al., 2014) have both been investigated using cryogenic MEG, they are still necessarily limited in the nature of the investigated social and physical interactions. Innovations in OPM-MEG allow for recording at room temperature, making it possible to design wearable systems (Boto et al., 2018) with the ability to investigate functional connectivity (Boto et al., 2021). Although these systems have been successfully used in adults, challenges regarding the signal-to-noise ratio (SNR), motion, and source localization remain and should be considered before this technique can be applied to the most pressing questions in infant neurodevelopment. The inability to use OPM-MEG in infants, and caregiver-infant dyads, remains a barrier to many urgent goals of developmental science, i.e., earlier identification of risk for developmental disorders and understanding the complex neural interplay that exists within the caregiver-infant dyad.

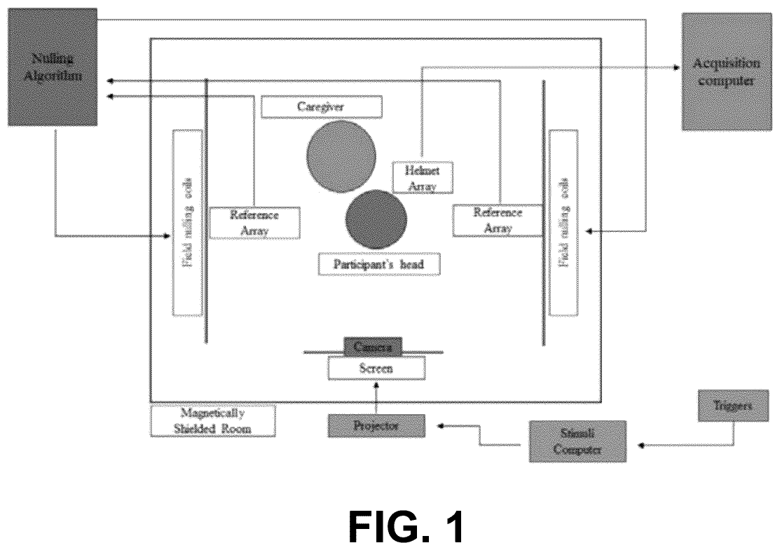

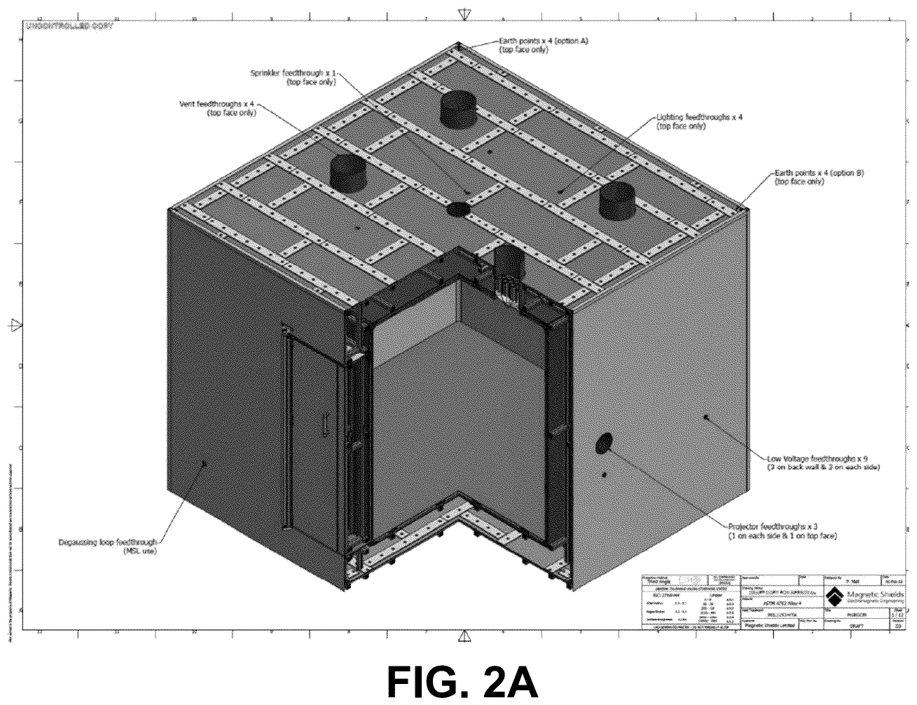

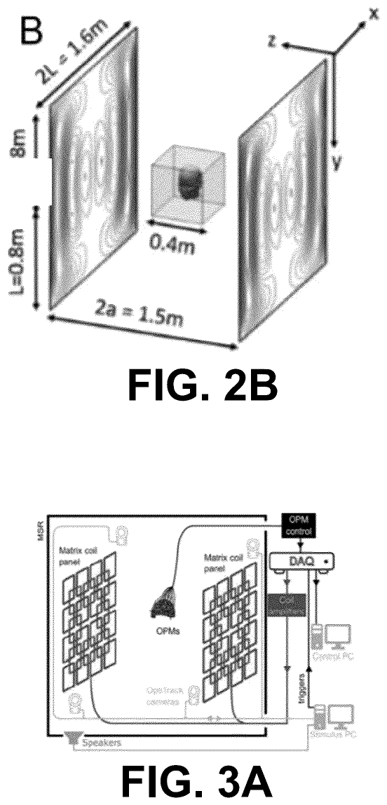

An overview of an OPM system configuration is depicted in FIG. 1. An optimal arrangement of an MSR (FIG. 2A) and participants (caregiver and infant) has been developed, and the use of static nulling coils has been optimized (Holmes et al. 2018). All of this has allowed a collection of the first ever OPM-MEG data in awake behaving infants between 3 and 8 months of age and an exploration of caregiver-infant dyads.

Structural MRIs: All MEG participants (adults and infants) will undergo a T1-weighted (adult) or both T1- and T2-weighted (infant) structural MRI. Scanning will be performed on a 3T Siemens Prisma (Erlangen, Germany) MRI scanner using a 32-channel Siemens head coil. T1 images for adults will be acquired with a 3D MPRAGE sequence (2 min, 34 s) with the following specifications: 1 mm isotropic voxel resolution; FOV of 256 mm (A-P)×247 mm (S-I)×176 mm (R-L); TI=950 ms; TR=1950 ms; TE=4.44 ms; FA=12 degrees; BW=140 Hz/pixel; GRAPPA parallel imaging factor of 4. Infant T1 data will be similarly acquired using a 3D MPRAGE sequence, but at 0.8 mm isotropic resolution, a field-of-view of 256 mm (A-P; phase), and the following scanning parameters: TI=1060 ms, TR=2400 ms, TE=2.24 ms, FA=8°, and GRAPPA factor of 2, for an acquisition time of 6 minutes and 38 seconds. The T2 will be acquired at 0.8 mm isotropic resolution, a field-of-view of 256 mm (A-P; phase) using a variable flip angle turbo spin-echo sequence with the following parameters: TR=3200 ms, TE=564 ms, and GRAPPA factor of 2, for an acquisition time of 5 minutes and 57 seconds. All infant scanning will be done during natural sleep without sedation.

### Experiment 1: Validate OPM-MEG Source Imaging During Parent-Infant Interactions

Experiment 1 aims to characterize OPM-MEG source localization in adults during caregiver-infant interactions by integrating triaxial OPM sensors and newly developed “matrix-coil” technology for spatially-adaptive active magnetic shielding within the MSR. The source imaging capabilities (i.e., source signal-to-noise ratio, SNR) of wearable OPM-MEG systems are limited by sensor noise and nonlinearities introduced by head movements within the non-zero background magnetic field of the MSR. Behavioral paradigms involving multiple persons interacting within the MSR will induce fluctuations in the MSR remnant field, which can further decrease sensor data quality caused by head movements. Source-space SNR will be evaluated during caregiver-infant interactions and changes in within-person source SNR due to the presence of multiple bodies within the MSR will be evaluated. It is possible that adult source-space SNR will be above the noise floor across multiple tasks while interacting with their infant and that SNR will be greater for one-person relative to multi-person experiments.

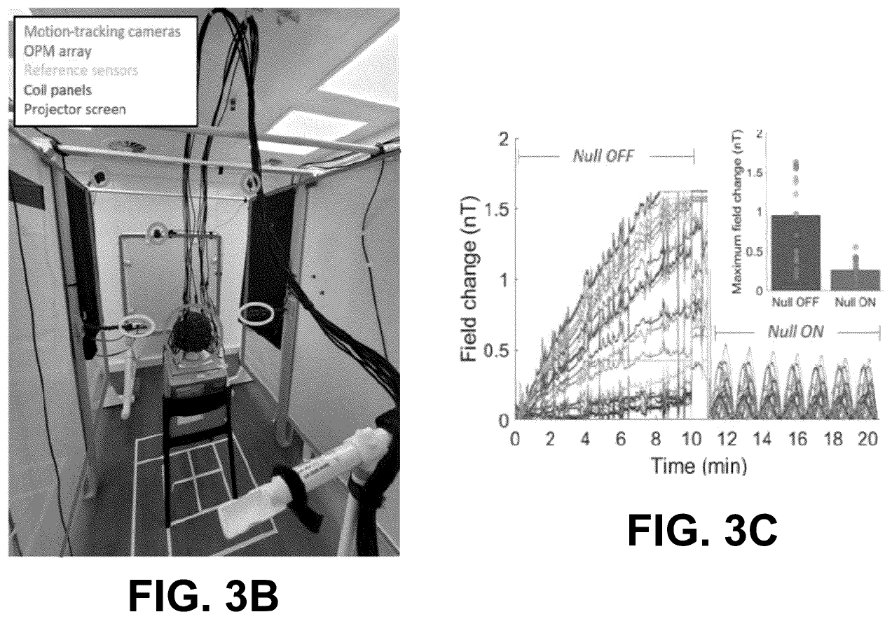

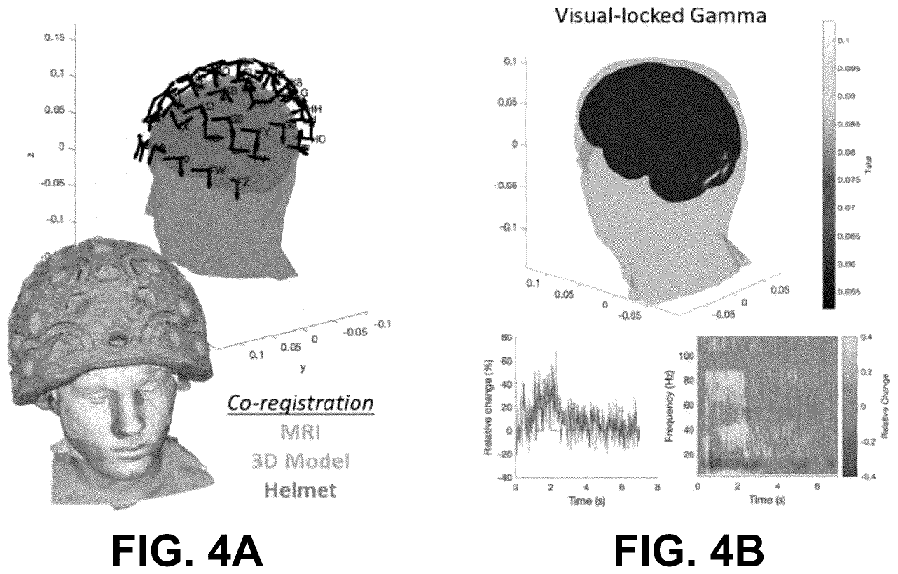

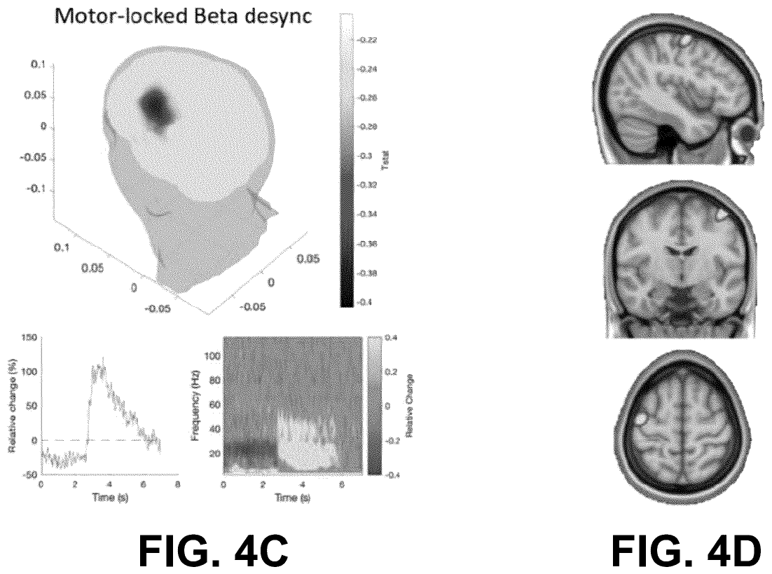

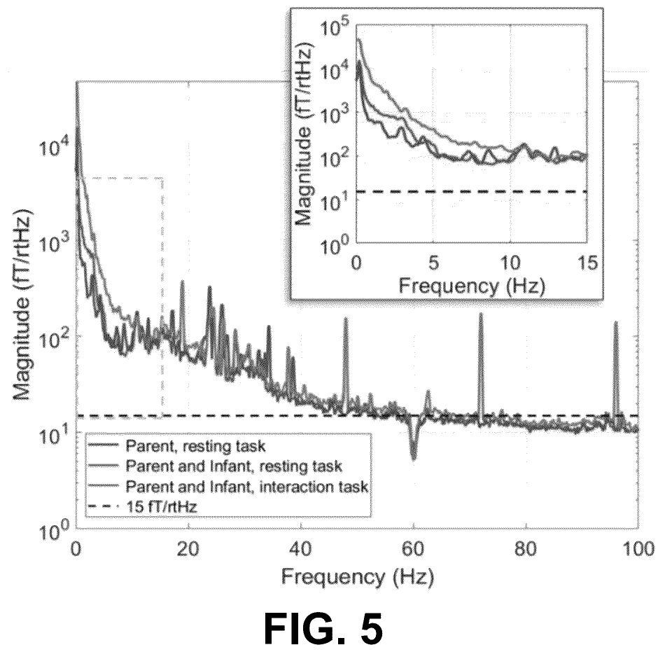

Two classes of well-validated tasks will be used, auditory evoked-response and frequency tagging tasks, and a visual-cued finger-tapping task. Electrophysiological activity during each task will be measured throughout the brain with a whole-head array of up to 50 triaxial Gen-3 QuSpin OPM sensors (see FIG. 3A). For the auditory evoked response and frequency tagging paradigms, the experiments of Corvilain et al., 2023 will be replicated and task parameters matched accordingly. The auditory evoked response task will involve presenting pure tones via projector speakers for 100 milliseconds with variable stimulus onset asynchrony (3+/−0.5 seconds); the auditory frequency tagging task will be based on an oddball paradigm where a sequence of pure tones is played (3 Hz pure tone rate) followed by an oddball tone (0.75 Hz oddball rate). These auditory tasks were selected for the adults given previous results using these paradigms showing their validity for use in infant OPM-MEG settings, thus ensuring similarity in tasks across adults and infants. For the visually-cued finger-tapping task (referred to as the visuomotor task), a paradigm similar to that of Boto et al., 2018 will be used in which a flashing visual stimulus (oval grating) cues the participant to tap their right index finger and to continue tapping their finger until the stimulus disappears. Each trial begins with the visual cue being presented for 2 s, followed by 5 s of rest before the next trial begins. This task robustly elicits a characteristic pattern of beta band (10-30 Hz) oscillatory power change in which beta band power decreases during finger movement followed by a “rebound” increase in power following cessation of the movement. Preliminary data for this visuomotor task using an array of 14 dual-axis (Y and Z) OPM sensors covering left motor and occipital cortices are shown in FIG. 4. The visuomotor task consists of 50 trials of visually-cued finger movements, and participants will complete the task 3 times. Head motion will be tracked during all tasks using an infrared camera system (OptiTrack V120 Duo; Corvallis, Oregon; see FIGS. 3A-3B). As control experiments, participants will also complete two 5-minute resting-state tasks, one task where participants are given instructions to play together as they might at home, allowing them to move freely while seated (interaction task), and one task where participants are instructed to focus on a crosshair projected onto a screen in the MSR (resting task). Participants will complete both the interaction and resting state tasks twice, once while alone in the MSR and once while interacting with their infant. Preliminary data for both resting and interaction tasks is shown in FIG. 5. Adult head position will be tracked throughout each experiment using an array of infrared motion-tracking cameras (FIG. 3B).

Generation-3 Triaxial Optically-pumped Magnetometers: The OPM-MEG cap uses the latest generation of OPM sensors from QuSpin (QuSpin, Inc.; Louisville, Colorado, USA), the “Gen-3” triaxial vector magnetometer (based on rubidium). The technology developmental space of miniaturized, commercially viable OPM sensors for high-density human MEG arrays has rapidly grown and advanced over recent years. The Gen-3 triaxial OPM from QuSpin measures all three spatial component vectors of magnetic fields (i.e., X, Y, and Z axes) compared to the previous Gen-2 dual-axis OPMs that measure only the radial (i.e., Z axis) and one tangential vector (i.e., Y axis) of the magnetic field relative to the scalp. The additional sensing axis of the Gen-3 triaxial OPMs provides an additional data channel with no increase in sensor density, maximizing the amount of information sampled. Also, by virtue of measuring the second tangential component vector of the magnetic field (i.e., the X axis), triaxial OPMs provide superior suppression of external interference, head-motion induced sensor distortions, and sensor cross-talk-related noise, which ultimately improves the quality of OPM-MEG source reconstruction (Brookes et al., 2022). Note that Gen-3 triaxial OPMs still have the same noise floor (15-20 ft/sqrt (Hz)) as the preceding generations of dual-axis OPMs as well as the same dynamic range (+/−5 nT), dimensions (12.4 mm×16.6 mm×24.4 mm), and vapor cell offset (6.5 mm) as the Gen-2 dual-axis OPMs. Furthermore, the Gen-3 triaxial OPM electronic controllers are backwards compatible with the preceding generations of dual-axis OPMs, enabling maximum integration of sensor arrays. The controllers are placed external to the MSR to minimize magnetic interference. The electronics controllers interface with the components of the OPM sensor: the laser light source, the photodiode, a pair of heating coils surrounding the vapor cell to prepare the rubidium gas in the spin-exchange relaxation free (SERF) regime, a set of three orthogonal axis magnetic coils surrounding the vapor cell for additional local field zeroing, and a set of modulation coils for establishing the directional sensing axes of the OPM (perpendicular to the direction of the light beam) and phase-sensitive lock-in signal amplification. The output of each OPM sensor is a voltage reading from the photodiode, acquired using a 16-bit DAQ system, that is proportional to the measured magnetic field. For all experiments, data are digitized at a sampling rate of 1200 Hz.

Sensor Positioning: The design and positioning of sensor arrays is one important component of wearable MEG systems based on OPMs. The rigid helmets, with known positions and orientations of OPM sensor slots, provide for robust recordings-sensors are in the same positions across participants, and do not move relative to one another during recordings- and have significant source reconstruction advantages compared to EEG-like flexible caps, demonstrating equivalent performance to cryogenic MEG-based source localization (Hill et al., 2020). Additionally, knowing the precise position and orientation of sensors from the rigid helmets allows for advanced data pre-processing steps for interference suppression like homogenous field correction (HFC; Tierney et al., 2021). This information is important for accurate active nulling of fluctuations in the ambient MSR field around the participant's head using bi-planar electromagnetic coils (Holmes et al., 2022, 2023a; Rea et al., 2021).

Sensor- and source-space SNR: Source-space SNR of the reconstructed time course is computed as the peak-to-peak change in the evoked response (signal) divided by the standard deviation in a baseline window following event offset (noise). The output-to-input SNR is then simply the source-space SNR value divided by the SNR of the best OPM-MEG sensor. Values greater than 1 indicate that the source reconstruction (i.e., beamformer) algorithm improves data quality, which requires both accurate forward models as well as accurate knowledge of the sensor array geometry and thus quantifies the overall performance of the OPM-MEG system. To identify peak source activity, a linearly-constrained minimum variance (LCMV) beamformer (van Veen et al., 1997; Boto et al., 2018) will be used to compute volumetric statistical parametric maps (SPMs) of the difference in spectral power between active and rest periods (visuomotor task; FIGS. 4B-4D) or between pre- and post-stimulus periods (auditory evoked response task, auditory oddball task). Source space and head models can be created based on individual's high-resolution structural MRI, the OPM array to individual brain anatomy can be co-registered (FIG. 4A), and a common forward model (Sarvas, 1987) can be used to compute the LCMV beamformer weights for a given voxel based on the sensor data covariance matrix during active and control task periods (Barnes and Hillebrand, 2003; Boto et al., 2018; FIGS. 4B-4C). The peak source activity voxel is chosen based on the peak difference in active versus control spectral power.

Magnetically Shielded Room: An MSR (FIG. 2A) built by Magnetic Shields, Ltd. to operate exceptionally in direct current (DC) and 0.01 Hz range will be used. The outer dimensions of the room are approximately 3734 mm×3734 mm×3124 mm (12.25 ft×12.25 ft×10.25 ft), and the inner dimensions are approximately 3001 mm×3001 mm×3124 mm (9.87 ft×9.87 ft×7.75 ft). The MSR, trademarked as a MuRoom®, uses magnetizable mu-metal to form a magnetically-shielded enclosure. The MuRoom is comprised of two layers of 1.5 mm mu-metal positioned external to a single layer of 8 mm aluminum with a 0.3 mm copper plating, followed by two more layers of 1.0 mm mu-metal internal to the single aluminum layer, for a total of four layers of mu-metal. For both pairs of two-layer mu-metal sheets, the individual mu-metal sheets are oriented orthogonally to one another to allow optimized grain structure directionality and to minimize gaps; all gaps between the mu-metal sheets are covered with 100 mm cover strips to further improve performance. The room is designed with apertures for airflow and projectors, and field leakage is reduced by using mu-metal sheets and mu-metal and aluminum branch tube covers to cover projector feedthroughs and air vents. The MuRoom is further outfitted with three electromagnetic coils around its exterior developed by Magnetic Shields, Ltd. to generate a highly homogenous field throughout the interior of the MSR. The geometry of the MuRoom coils is optimized for ultra-low-field magnetic resonance experiments. Furthermore, keeping the background magnetic field of the MuRoom as homogenous as possible via actively shielding (i.e., degaussing) with electromagnetic coils is critical for sustaining performance of OPM sensors based on cesium, rubidium, or potassium, primarily by permitting a maintainable polarization state of the nuclear-spin polarized substance. The average maximum field change measured by an array of 12 dual-axis OPM sensors placed in the center of the MuRoom over the course of 10 minutes was 0.95 nT, with a standard deviation of 0.57 nT (FIG. 3C).

Active Nulling of Ambient MSR Field Fluctuations: OPM sensors used for wearable human MEG require near-zero Tesla background magnetic fields to operate. The ambient field within the MSR, even with passive shielding using the MuRoom's walls and surrounding electromagnetic coils, is never truly equal to zero Tesla. Furthermore, although the remnant field within the Virginia Tech MuRoom after degaussing is ˜1.4 nT, head movements of the participant through the remnant field can still greatly distort and even saturate OPM sensor outputs. To keep OPM sensors working optimally, it is often required to integrate additional active shielding using large, bi-planar electromagnetic coils positioned on either side of a participant within the MSR (FIGS. 3A-3B; Boto et al., 2018; Holmes et al., 2018, 2019, 2023a, 2023b). Such electromagnetic coils are controlled to emit a magnetic field that is equal and opposite to field measurements of reference sensors placed close to the participant's head (FIG. 3B), creating a low, spatially homogenous field in the area surrounding the OPM array. The precise spatial region nulled by the matrix coils can be configured to always surround the OPM array during an experiment by using real-time head motion tracking to determine the relative position of the participant's head to the coils, and from there deciding which component coils of the matrix coils to activate for shielding (Holmes et al., 2023a). The optimal currents to input to the matrix coil determined at each moment are based on field measurements from on-scalp reference sensors, thus providing spatially-adaptive and stable active nulling. Note that preliminary data were collected using a set of bi-planar electromagnetic coils (FIG. 3B) that produce a spatially homogenous magnetic field in a fixed 40 cm×40 cm×40 cm cube in the center of the two coils (Boto et al., 2018; Holmes et al., 2018). These coils significantly reduce the absolute mean field changes across an array of OPM sensors placed in the center of the MSR to 0.25 nT and prevent sensor gain nonlinearities. The novel matrix coil design has been shown to provide similar active nulling capabilities to the preceding version of the coils that produce a spatially-fixed nulled region (Holmes et al., 2023a).

### Experiment 2: Sensor Mounting Tests

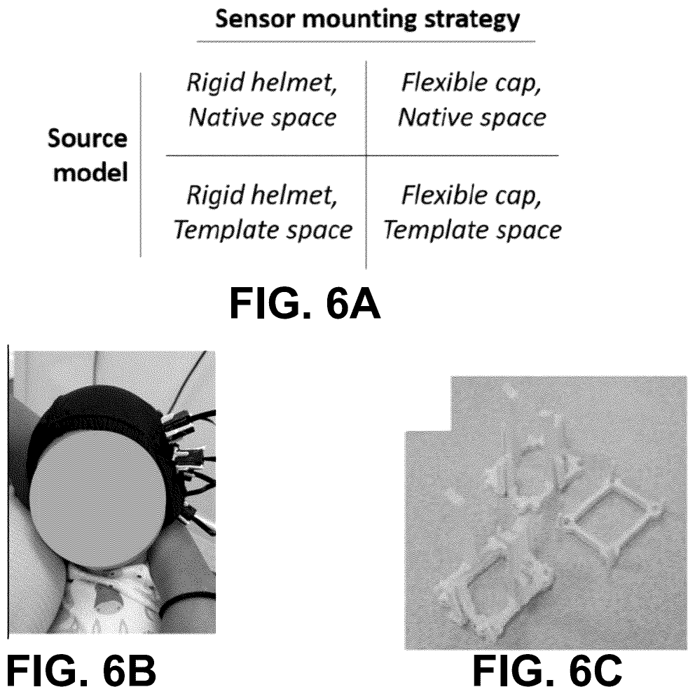

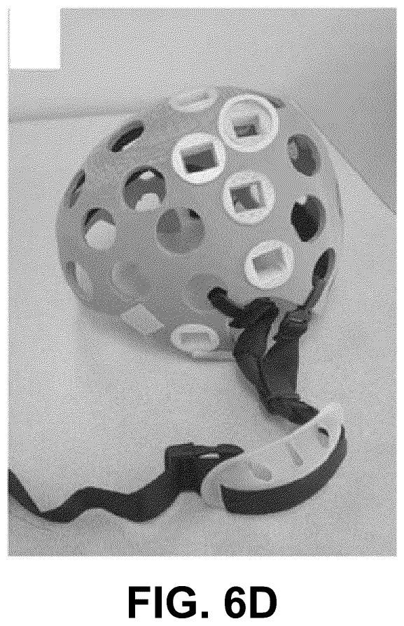

In pursuit of developing wearable OPM-MEG systems into a lifespan compliant functional neuroimaging technology, previous experiments have relied on either creating different rigid helmets adapted for certain age ranges (e.g., adult helmets, 2-year-old helmet, 4-year-old helmet, etc.) or using flexible, EEG-like caps with 3D-printed sensor holders (Hill et al., 2019, 2020). While both rigid helmets and flexible caps are viable solutions for mounting OPM sensors on the scalp, each method has fundamental limitations that must be considered when optimizing sensor array designs for accessibility and scalability. Rigid helmets must be additively manufactured and do not allow for flexible sensor placement once printed but do provide accurate knowledge of relative sensor positions and orientations with respect to the helmet. Flexible caps allow for flexible sensor placement, are easily adjustable to individual anatomy, and do not require costly additive manufacturing, but are lacking in information regarding sensor position and orientation. Furthermore, whereas sensors are fixed and therefore do not move relative to one another in rigid helmets, flexible caps permit individual sensors to move independently which contributes to random distortions in sensor data and co-registration errors that can decrease the robustness of source reconstruction (Hill et al., 2019). These primary and secondary problems with current sensor mounting solutions are magnified in the context of freely-moving infant OPM-MEG recordings given the uncontrollable and large head and body movements of infants. Previous studies developing neonatal OPM-MEG have opted for the flexible cap design and for swaddling the neonate during OPM-MEG recording to further buffer against motion-induced sensor distortions (see FIG. 6B, adapted from Feys et al., 2023; see also Corvilain et al., 2023), which is a common approach for pediatric functional neuroimaging using MEG (Clarke et al., 2022). However, swaddling to reduce motion in awake infants quickly becomes ineffective as the infant grows, thus there remains a basic need for developing sensor mounting strategies for infant OPM-MEG that provide robust source reconstruction performance even during free motion. A method for 3D printing rigid helmets for infant OPM-MEG is disclosed herein. The helmets are lightweight, well tolerated, and provide secured positioning of OPM sensors close to the infant scalp (˜6 mm scalp-to-sensor distance; FIG. 6D).

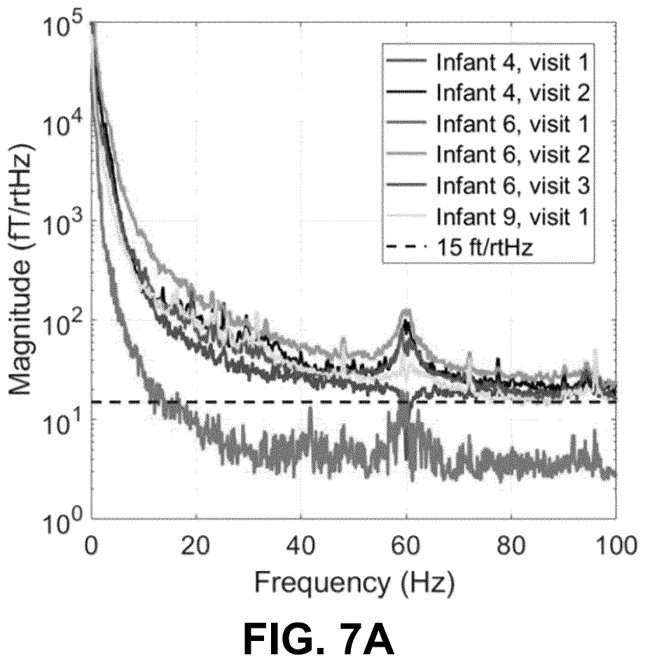

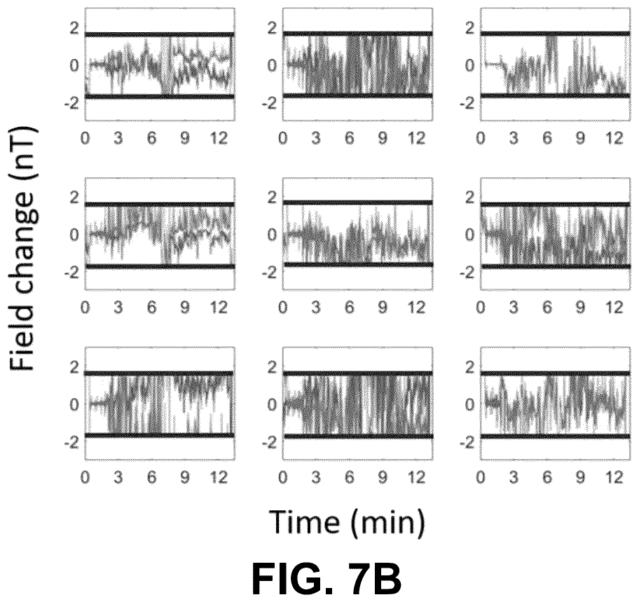

Sensor mounting: Four different combinations of sensor mounting strategies and source models of infant anatomy in a 2×2 design (see FIG. 6A) will be evaluated. For the 3D-printed rigid helmets, sensors are mounted onto a number of pre-defined sensor positions (FIG. 6D) using custom 3D-printed sensor slot inserts and held at a fixed orientation using additional sensor holders (FIG. 6C). For the flexible caps, a setup similar to Feys et al., 2023 will be used, with the same sensor holders as in the rigid helmets (FIG. 6C) to mount OPM sensors onto flexible caps designed for infants (EasyCap, Worthsee, Germany). For experiments using either rigid helmets or flexible caps, 20 Gen-3 triaxial OPM sensors will be used. Preliminary data (FIG. 7) indicate that freely-moving infants tolerate a moderate number of sensors (e.g., range 8-13 sensors for data in FIG. 7). First, flexible caps, like the one shown in FIG. 6B, will be used to collect OPM-MEG recordings from freely-moving infants and to perform source reconstruction using the infant's native MRI anatomy derived from combined T1- and T2-weighted structural MRI scans. Next, rigid helmets will be used to collect infant OPM-MEG data and native source model for source reconstruction. Finally, the rigid helmet will be used to mount OPM sensors and an infant template brain will be used as the source model for source reconstruction.

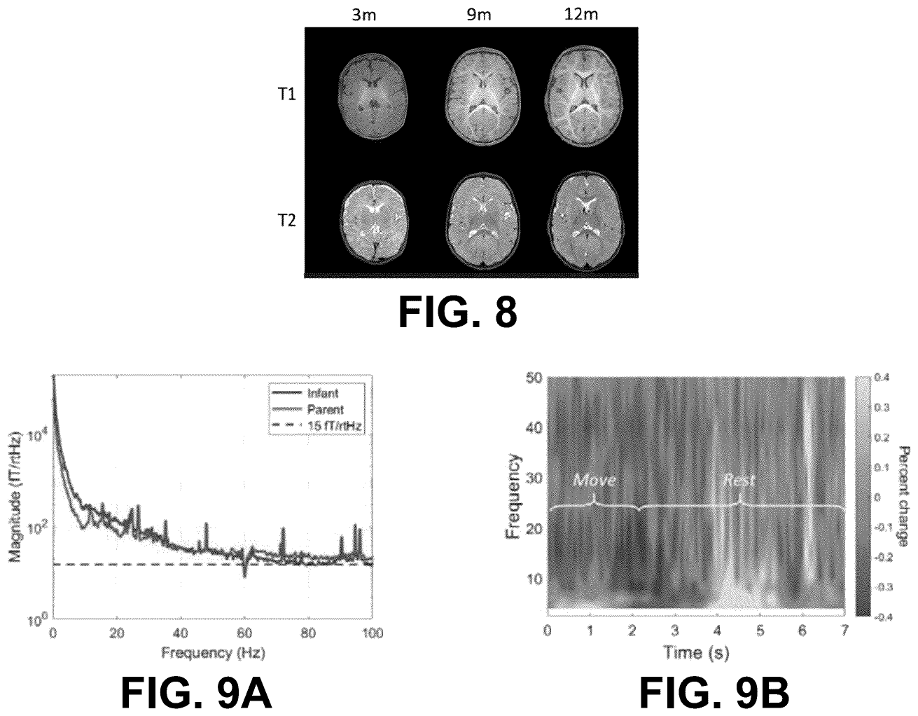

Structural MRIs: Both the T1 and T2 images (FIG. 8) will be used to co-register the OPM-MEG data. Established infant segmentation pipelines will be applied to automatically identify tissue classes, cortical, and subcortical regions (Hendrickson et al. 2023).

OPM-MEG and MSR: Infant head motion will be tracked using motion-tracking cameras and integrated with the matrix coil technology for spatially-precise active shielding in the region surrounding the infant head. The preliminary data was collected using a set of bi-planar electromagnetic coils that produce a spatially-fixed homogenous field in a 40 cm cube within the center of the MSR. OPM sensors will be operated in lowered gain modes to better withstand infant head movements. The Gen-3 QuSpin OPM sensor output voltage at the standard sensor gain is proportional to the measured magnetic field at 2.7 V/nT, but infant head movements can often cause OPM sensors to experience field changes >1.85 nT which exceed their rail-to-rail dynamic operating range (+/−5 V), causing sensors to saturate. Lowering the gain on OPM sensors to 0.9 V/nT effectively widens the dynamic range of field levels that OPM sensors can measure (+/−5.5 nT) without saturating their output, but at the cost of increasing broadband noise. Preliminary data in FIG. 7 was collected at standard OPM gain (2.7 V/nT). A lowered analog output gain setting (0.9 V/nT) can also be used.

Sensor- and source-level SNR: Output-to-input SNR can be used as the primary measure of rigid helmet and flexible cap performance. Rigid helmets are more likely to demonstrate improved source-level SNR compared to a flexible cap by virtue of providing more robust and precise sensor position and orientation information to improve source modeling relative to flexible caps. Co-registration of sensor positions from the rigid helmets and flexible caps to infant neuroanatomy will be accomplished using an optical 3D-modeling approach (see FIG. 4A). To supplement the optical co-registration procedures, using the bi-planar electromagnetic coils to apply known magnetic fields across the OPM array will also be explored, thereby enabling identification of the relative positions and orientations of each sensor in the array based on its response to the applied field (livanainen et al., 2022). For both sensor mounting strategies, sensor positions will be co-registered relative to both an infant's native brain source space (derived from T1- and T2-weighted MRI volumes, FIG. 8) as well as to an age-appropriate template model (Chen et al. 2022). Output-to-input SNR will be computed using both native and template source spaces to characterize changes in SNR due to individually-precise source spaces for forward modeling, but it is likely that there will be no significant changes in output-to-input SNR when using native or template source spaces.

Tasks: Experiment 2 will use the same resting state, auditory, and visuomotor tasks used for Experiment 1. Behavioral scoring of video recordings of the experiment will be used to indicate segments where the infant was attentive to visual stimuli during the visuomotor task. Furthermore, during the visuomotor task, the caregiver will be responsible for tapping the right hand of the infant when visually cued by the task. Otherwise, the infant will be allowed to move freely while supported upright by their caregiver who will be seated in the center of the MSR.

### Experiment 3: Motion-Tolerant Parent-Infant Hyperscanning

Motion-tolerant caregiver-infant hyperscanning experiments will be established by combining spatially-adaptive matrix coil active shielding technology with real-time algorithms for dynamic field correction of sensor distortions caused by head motion. The primary outcome measure will be output-to-input SNR of auditory and sensorimotor evoked responses. However, simultaneous OPM-MEG recordings will also be involved, using sensor arrays mounted on both the infant and caregiver heads, with source reconstruction SNR being evaluated in both infants and caregivers. The caregiver-infant dyad will be seated and positioned in the center of the MSR between the bi-planar electromagnetic coils. The capability of matrix coil magnetic shielding technology for nulling background MSR field fluctuations around the heads of both the caregiver and infant at the same time. One prior study has demonstrated the ability of the matrix coil technology to reduce ambient field levels surrounding two adults performing hand-touching and paddle ball tasks during OPM-MEG hyperscanning (Holmes et al., 2023a). However, participants in this study were MEG experts and adults who were instructed to not make large movements during the experiments. Developing the same matrix coil technology for caregiver-infant hyperscanning, in which correlated movements between the caregiver and infant are unpredictable, will represent a large step forward towards adapting OPM-MEG hyperscanning paradigms to other populations with uncontrollable movements (e.g., tremor disorders).

Relatedly, given the large amount of body motion during caregiver-infant interactions, testing will also be done on emerging methods for dynamic field correction of OPM sensor distortions caused by head movements within a background magnetic field (Robinson et al., 2022; Mellor et al., 2023). Given that the background MSR field is never truly zero, head movements distort OPM sensor measurements and can cause gain alterations and sensor saturation related to cross-axis projection errors (Borna et al., 2022). Dynamic field compensation is similar in theory to the matrix coil active shielding technology that nulls ambient MSR field fluctuations, with the key difference being that dynamic field compensation algorithms do not alter the MSR background field but instead use software to directly alter onboard OPM modulation coils to actively cancel-out head motion-related distortions with low latency. Recent approaches to applying dynamic field correction during OPM-MEG experiments have used transformed direct field measurements based on an orthogonal reference sensor array (Robinson et al., 2022) or generative models (i.e., spherical harmonics; Mellor et al., 2023) to determine appropriate compensation currents to apply to OPM sensors during movements or fluctuations in the background field. These experiments will combine dynamic field compensation methods with spatially-adaptive external magnetic shielding to establish motion-tolerant OPM-MEG recordings for both adults and infants. Combining both methods for active field control will establish motion-tolerant OPM-MEG recordings through (i) ensuring active shielding of ambient field fluctuations (matrix coil) and (ii) using online dynamic field correction to mitigate OPM sensor distortions caused by movements or fluctuations in the known ambient field. Using the same behavioral paradigms as Experiment 1, the performance of dynamic field correction methods and spatially-adaptive active shielding for OPM-MEG hyperscanning will be evaluated using measures of both parent and infant SNR.

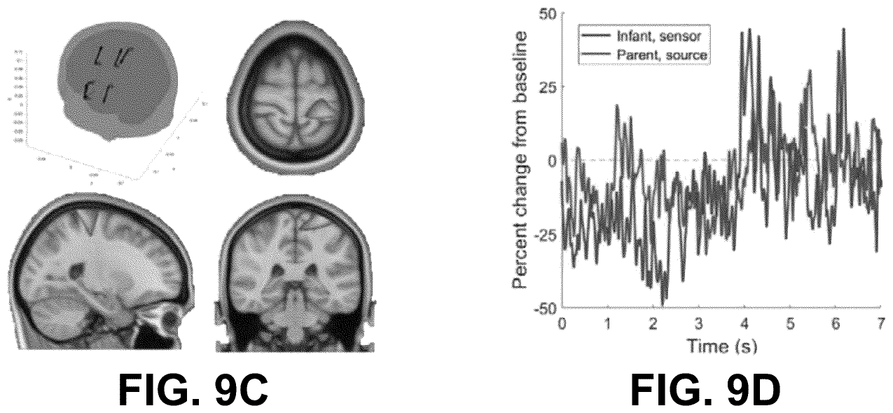

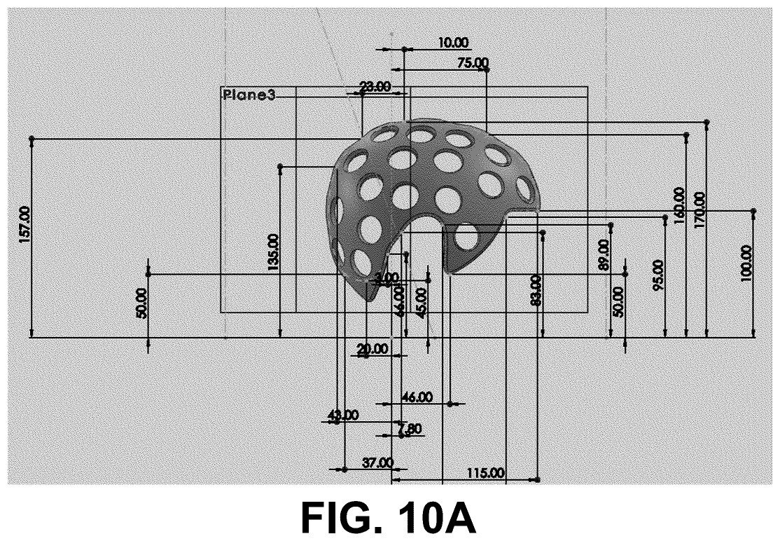

Tasks: Experiment 3 will use the same resting-state, caregiver-infant interaction, auditory stimulation, and visuomotor paradigms as Experiments 1 and 2. For the resting-state task (5 minutes), the caregiver will be instructed to fixate on a crosshair presented in the center of the projection screen. For the caregiver-infant interaction task (5 minutes), the caregiver and infant will be instructed to interact and move normally. The auditory stimulation paradigms (6 minutes) will be the same as in other experiments. The visuomotor task (6 minutes) will involve the caregiver holding the right hand of the infant with the index finger and thumb of their right hand (or the left hand of the caregiver holding the left hand of the infant) and pressing/tapping the infant's hand when cued by the visual stimulus. See FIGS. 9A-9D for preliminary results of the visuomotor caregiver-infant hyperscanning task.

OPM-MEG and MSR: The experiments of experiment 3 will use the same triaxial OPM sensors and matrix coil technology within the MSR as experiments 1 and 2. The one previous OPM-MEG hyperscanning study involved two adults collected MEG data using the lower analog output gain of the triaxial OPM sensors (0.9 V/nT) so that the sensors could withstand a wider range of field magnitude to be expected during multi-person experiments, resulting in accurate source reconstruction of sensorimotor activity (Holmes et al., 2023a). The pilot experiment of a mother-infant hyperscan recording (FIGS. 9A-9D) also used the lower analog output gain for an array of 12 dual-axis Gen-2 OPMs, with 6 sensors each placed over the mother's and infant's left sensorimotor cortex. Preliminary results demonstrate the potential for source imaging of both parents and infants for the study of physical and social interactions across the lifespan. Still, operating OPM sensors in lower gain regimes compromises on sensitivity. Experiment 3 proposes to evaluate using dynamic field correction methods to compensate for head-motion induced sensor distortions, thereby enabling the operation of OPM sensors at standard gain levels to maximize sensitivity during ambulatory or multi-person interaction experiments.

Sensor positioning and two-person recordings: Similar to Experiments 1 and 2, generic rigid caps will be used to mount whole-scalp-covering sensor arrays for infant and adult OPM-MEG recordings. 35 triaxial OPM sensors will be used for the adult array and 15 triaxial OPM sensors for the infant array. The infant and adult OPM arrays will be controlled using two separate 16-bit DAQ systems. A combination of OPM sensors from infant and adult arrays will be used for calibrating the matrix coil technology so that a greater number of component coils are activated for spatially-precise nulling around both infant and adult heads, thus providing for stronger nulling capabilities. To apply dynamic field compensation, a small number of scalp-mounted OPM sensors from the infant array will be used to define the corrections applied to other OPM sensors on the infant array; OPM sensors from the adult array will be used to define the compensation currents for other OPM sensors on the adult array.

Source SNR: Source-space SNR will be used as the primary measure of assessing the ability to reconstruct source activity in both caregivers and infants during OPM-MEG hyperscanning. Source reconstruction will be conducted using native source spaces for both adults and infants derived from structural MRI scans. As a secondary analysis, the output-to-input SNR for both adults and infants with dynamic field correction applied or not will be assessed, with the null hypothesis being no changes in SNR.

### SUMMARY

The experiments disclosed herein tackle some of the most difficult issues facing wearable OPM-MEG systems head-on through the use of caregiver-infant interaction paradigms to accelerate establishing OPM-MEG systems for the study of human social interactions across the lifespan. The work will serve as benchmarks for future OPM-MEG system development for other human populations with uncontrollable movements, or for behavioral paradigms with unpredictable body motion.

### REFERENCES FOR EXAMPLE 1

1. Boto E, Holmes N, Leggett J, Roberts G, Shah V, Meyer S S, Muñoz LD, Mullinger K J, Tierney T M, Bestmann S, Barnes G R, Bowtell R, and Brookes M J. (2018). Moving magnetoencephalography towards real-world applications with a wearable system. Nature 555:657-661.
2. Holmes N, Rea M, Hill R M, Boto E, Leggett J, Edwards L J, Rhodes N, Shah V, Osborne J, Fromhold T M, Glover P, Montague P R, Brookes M J, and Bowtell R. (2023a). Naturalistic hyperscanning with wearable magnetoencephalography. Sensors 23 (12): 5454. doi: 10.3390/s23125454
3. Brookes M J, Leggett J, Rea M, Hill R M, Holmes N, Boto E, and Bowtell R. (2022). Magnetoencephalography with optically pumped magnetometers (OPM-MEG): the next generation of functional neuroimaging. Trends in Neurosciences 45 (8): 621-634.
4. Hill R M, Boto E, Holmes N, Hartley C, Seedat Z A, Leggett J, Roberts G, Shah V, Tierney T M, Woolrich M W, Stagg C J, Barnes G R, Bowtell R, Slater R, and Brookes M J. (2019). A tool for functional neuroimaging with lifespan compliance. Nature Communications 10 (1): 1-11.
5. Corvilain P, Wens V, Bourguignon M, Capparini C, Fourdin L, Ferez M, Feys O, De Tiege X, and Bertels 5 (2023). Extending the Applicability of Optically Pumped Magnetoencephalography toward Early Human Life. bioRxiv. doi: 10.1101/2023.10.28.564455.
6. Feys O, Corvilain P, Aeby A, Sculier C, Holmes N, Brookes M, Goldman S, Wens V, and De Tiège X. (2022). On-Scalp Optically Pumped Magnetometers versus Cryogenic Magnetoencephalography for Diagnostic Evaluation of Epilepsy in School-Aged Children. Radiology 304 (2): 429-34.
7. Howell B R, Styner M A, Gao W, Yap P-T, Wang L, Baluyot K, Yacoub E, Chen G, Potts T, Salzwedel A, Li G, Gilmore J H, Piven J, Smith J K, Shen D, Ugurbil K, Zhu H, Lin W, and Elison J T. (2019). Then UNC/UMN Baby Connectome Project (BCP): An overview of the study design and protocol development. Neurolmage 185:891-905.
8. Spann M N, Wisnowski J L, Ahtam B, Gao W, Huang H, Nebel M B, Norton E S, Ouyang M, Rajagopalan V, Riggins T, Saygin Z M, Scott L, Smyser C D, Thomason M E, Wakschlag L S, Ahmad S, Aydin E, Barkovich A J, Berger-Jenkins E, Brick J, Bowman L C, Camacho M C, Lugo-Candelas C, Cusack R, DuBois J, Dufford A J, Elison J T, Ellis C T, Ferradal S L, Filippi C, Ford A L, Fouladivanda M, Gaab N, Gano D, Ganz-Benjaminsen M, Ghetti S, Glenn O A, Gomez M J C, Graham A, Hendrix C L, Holland C M, Humphreys K, Korom M, Kosakowski H L, Li G, Manessis A G, Nolvi S, Pineda R, Pollatou A, Rae C, Rasmussen J M, Scheinost D, Shultz S, Simon-Martinez C, Madsen K S, Sung S, Sylvester C M, Turesky T K, Vaughn K A, Wagner L, Wang L, Warton F L, Wilson S, Wintermark P, Wu Y, Yap P-T, Yates T S, Yen E, Yu X, Zhu H, Zöllei L, Howell B, and Dean D C. (2023). The art, science, and secrets of scanning young children. Biological Psychiatry 93 (10): 858-860.
9. Borna A, Carter T R, Colombo A P, Jau Y-Y, Mckay J, Weisend M, Taulu S, Stephen J M, Schwindt P D D. (2020). Non-invasive functional brain imaging with an OPM-based magnetoencephalography system. PLOS One 15 (1): e0227684.
10. Robinson S E, Andonegui A B, Holroyd T, Hughes K J, Alem O, Knappe S, Maydew T, Griesshammer A, and Nugent A. (2022). Cross-axis dynamic field compensation of optically pumped magnetometer arrays for MEG. Neurolmage 262:1-12.
11. Mellor S, Tierney T M, Seymour R A, Timms R C, O'Neill G C, Alexander N, Spedden M E, Payne H, and Barnes G R. (2023). Real-time, model-based magnetic field correction for moving, wearable MEG. Neurolmage 278:1-13.
12. Shultz S, Klin A, and Jones W. (2018). Neonatal Transitions in Social Behavior and Their Implications for Autism. Trends in Cognitive Science 22:452-469.
13. Hadders-Algra, M. (2021). Early diagnostics and early intervention in neurodevelopmental disorders-age-dependent challenges and opportunities. Journal of clinical medicine 10 (4): 61.
14. Webb S J, Jones E J H, Kelly J, and Dawson G. (2014). The motivation for very early intervention for infants at high risk for autism spectrum disorders. International Journal of Speech-Language Pathology 16:36-42.
15. Chen Y H, Saby J, Kuschner E, Gaetz W, Edgar J C, and Roberts T P L. (2019).

Magnetoencephalography and the infant brain. Neurolmage 189:445-458.
16. Boto E, Seedat Z A, Holmes N, Leggett J, Hill R M, Roberts G, Shah V, Fromhold T M, Mullinger K J, Tierney J. (2019). Wearable neuroimaging: Combining and contrasting magnetoencephalography and electroencephalography. Neurolmage 201:116099.
17. Hill R M, Boto E, Rea M, Holmes N, Leggett J, Coles L A, Papastavrou M, Everton S K, Hunt B A E, Sims D, Osborne J, Shah V, Bowtell R, and Brookes M J. (2020). Multi-channel whole-head OPM-MEG: helmet design and a comparison with a conventional system. Neurolmage 219:116995.
18. Hämäläinen M, Hari R, Ilmoniemi R J, Knuutila J, and Lounasmaa O V. (1993). Magnetoencephalography theory, instrumentation, and applications to noninvasive studies of the working human brain. Reviews of Modern Physics 65:413-497.
19. Lew S, Sliva D D, Choe M S, Grant P E, Okada Y, Wolters C H, and Hämäläinen MS. (2013). Effects of sutures and fontanels on MEG and EEG source analysis in a realistic infant head model. Neurolmage 76:282-293.
20. Hirata M, Ikeda T, Kikuchi M, Kimura T, Hiraishi H, Yoshimura Y, and Asada M. (2014). Hyperscanning MEG for understanding mother-child cerebral interactions. Frontiers in Human Neuroscience 8:118.
21. Boto E, Hill R M, Rea M, Holmes N, Seedat Z A, Leggett J, Shah V, Osborne J, Bowtell R, and Brookes M J. (2021). Measuring functional connectivity with wearable MEG. Neurolmage 230:117815.
22. Holmes N, Leggett J, Boto E, Roberts G, Hill R M, Tierney T M, Shah V, Barnes G R, Brookes M J, and Bowtell R. (2018). A Bi-Planar Coil System for Nulling Background Magnetic Fields in Scalp Mounted Magnetoencephalography. Neurolmage 181:760-74.
23. Boto E, Shah V, Hill R M, Rhodes N, Osborne J, Doyle C, Holmes N, Rea M, Leggett J, Bowtell R, and Brookes M J. (2022). Triaxial Detection of the Neuromagnetic Field Using Optically-Pumped Magnetometry: Feasibility and Application in Children. Neurolmage 252:119027.
24. Holmes N, Rea M, Hill R M, Leggett J, Edwards L J, Hobson P J, Boto E, Tierney T M, Rier L, Rivero G R, Shah V, Osborne J, Fromhold T M, Glover P, Brookes M J, and Bowtell R. (2023b). Enabling ambulatory movement in wearable magnetoencephalography with matrix coil active shielding. Neurolmage 274:120157.
25. Tierney T M, Alexander N, Mellor S, Holmes N, Seymour R, O'Neill G C, Maguire E A, and Barnes G R. (2021). Modelling optically pumped magnetometer interference in MEG as a spatially homogenous magnetic field. Neurolmage 244:118484.
26. Holmes N, Rea M, Chalmers J, Leggett J, Edwards L J, Nell P, Pink S, Patel P, Wood J, Murby N, Woogler D, Dawson E, Mariani C, Tierny T M, Mellor S, O'Neill G C, Boto E, Hill R M, Shah V, Osborne J, Pardington R, Fierlinger P, Barnes G R, Glover P, Brookes M J, and Bowtell R. (2022). A lightweight magnetically shielded room with active nulling. Scientific Reports 12 (1): 13561.
27. Rea M, Holmes N, Hill R M, Boto E, Leggett J, Edwards L J, Woogler D, Dawson E, Shah V, Osborne J, Bowtell R, and Brookes M J. (2021). Precision magnetic field modelling and control for wearable magnetoencephalography. Neurolmage 241:118401.
28. Van Veen B D, Van Drongelen W, Yuchtman M, and Suzuki A. (1997). Localization of brain electrical activity via linearly constrained minimum variance spatial filtering. IEEE Transactions on Biomedical Engineering 44 (9): 867-880.
29. Sarvas J. (1987). Basic mathematical and electromagnetic concepts of the biomagnetic inverse problem. Physics in Medicine and Biology 32 (1): 1-11.
30. Barnes G R and Hillebrand A. (2003). Statistical flattening of MEG beamformer images. Human Brain Mapping 18 (1): 1-12.
31. Holmes N, Tierney T M, Leggett J, Boto E, Mellor S, Roberts G, Hill R M, Shah V, Barnes G R, Brookes M J, and Bowtell R. (2019). Balanced, bi-planar magnetic field and field gradient coils for field compensation in wearable magnetoencephalography. Scientific Reports 9:14196.
32. Feys O, Corvilain P, Bertels J, Sculier C, Holmes N, Brookes M, Wens V, and De Tiege X. (2023). On-scalp magnetoencephalography for the diagnostic evaluation of epilepsy during infancy. Clinical Neurophysiology 155:29-31.
33. Clarke M D, Bosseler A N, Mizrahi J C, Peterson E R, Larson E, Meltzoff A N, Kuhl P K, and Taulu S. (2022). Infant brain imaging using magnetoencephalography: Challenges, solutions, and best practices. Human Brain Mapping 43:3609-3619.
34. Hendrickson T J, Reiners P, Moore L A, Perrone A J, Alexopoulos D, Lee E G, Styner M, Kardan O, Chamberlain T A, Mummaneni A, Caldas H A, Bower B, Stoyell S, Martin T, Sung S, Fair E, Uriarte-Lopez J, Rueter A R, Yacoub E, Rosenberg M D, Smyser C D, Elison J T, Graham A, Fair D A, and Feczko E. (2023). BIBSNet: A Deep Learning Baby Image Brain Segmentation Network for MRI Scans. bioRxiv. doi: 10.1101/2023.03.22.533696.
35. livanainen J, Borna A, Zetter R, Carter T R, Stephen J M, Mckay J, Parkkonen L, Taulu S, Schwindtt P D D. Calibration and localization of optically pumped magnetometers using electromagnetic coils. Sensors 22 (8): 3059.
36. Chen L, Wu Z, Hu D, Wang Y, Zhao F, Zhong T, Lin W, Wang L, and Li G. (2022). A 4D Infant Brain Volumetric Atlas Based on the UNC/UMN Baby Connectome Project (BCP) Cohort. Neurolmage 253:119097.
37. Borna A, livanainen J, Carter T R, Mckay J, Taulu S, Stephen J, Schwindtt P D D. (2022). Cross-axis projection error in optically pumped magnetometers and its implications for magnetoencephalography systems. Neurolmage 247:118818.
38. Ciesielski K R and Stephen J M. (2014). Pediatric MEG: Investigating spatio-temporal connectivity of developing networks. In S. Supek and C. J. Aine (Eds.), Magnetoencephalography: From Signals to Dynamic Cortical Networks (pp. 525-555). Springer Berlin, Heidelberg.

### Example 2

Cerebral Palsy (CP) is the most common motor disorder experienced in childhood with an estimated prevalence of 3 per 1,000 births. Early detection is crucial in gaining access to intervention, and guidelines recommend the use of structural MRI combined with functional motor assessment. However, there is a methodological disconnect that impacts current understanding of the relationship between early brain function and motor impairment, representing a major barrier to improving outcomes for infants at high risk of neuromotor impairment.

A multi-modal functional neuroimaging approach could be used to characterize the relationships between intrinsic brain activity and motor abilities in infants. A multi-modal functional imaging approach includes using both resting state functional MRI (rsfMRI) and a recent innovation in the field of magnetoencephalography that uses OPM-MEG.

The integration of both MRI and OPM-MEG offers an innovate and powerful tool set for investigating the intricate relationship between early brain function and motor development, which can ultimately lead to earlier diagnosis and improved outcomes for children with motor dysfunction.

### OPM-MEG Facilities

OPMs are room-temperature, small-footprint, magnetic field sensors that can be used for magnetoencephalography (OPM-MEG). These units require a very low background magnetic field and enclosure to function. Examples of OPMs that can be used for OPM-MEG include 20-second-generation QuSpin OPMs. These sensors have a sensitivity of 7-10 fT/√Hz, a bandwidth of 135 Hz, and can operate with a background of 200 nT. The sensors have a footprint of only 12.4×16.6×24.4 mm, weigh 4 g, and have a surface temperature of ˜41° C., allowing them to be located 6.5 mm from the scalp, in possibly very lightweight headgear, making them ideal for MEG experiments in conscious human participants. An ultra-low-field room can be used in human neuroimaging via OPM-MEG. These magnetically shielded rooms can be constructed from highly magnetizable mu-metal (such as mu-metal walls) and will be equipped with a stimulus projector and button boxes for human imaging experiments. For a complete description of the room see the facilities page. The OPM-MEG facility also has button boxes, cameras, a microphone, and MEG compatible speakers for stimulus delivery.

### Preliminary Data

RsfMRI provides excellent spatial resolution but is limited in temporal resolution based on hemodynamic response time, as well as sensitivity to even very small movements that do not make this a feasible modality to collect awake motor task data for infant subject. In contrast, MEG has excellent temporal resolution that provides insight into questions about the precise timing of neural activity to be synced with awake behaviors. With recent innovations in OPM-MEG participants can be scanned in a variety of positions while doing various tasks, making it an attractive modality for its utility in young children. An OPM-MEG cap has been developed that provides whole brain coverage and customizability for a range of infant head shapes and sizes, all while being lightweight and tolerable for infant use. Fifteen (15) infant OPM sessions have been completed using a variety of stimuli including visual, auditory, and tactile tasks. The preliminary results support the feasibility of the novel methodology for infant use.

### Materials and Methods

Pictures of the subject's (infant) face from the side and front will be obtained. This information can be used to create a custom 3D-printed MEG cap that will be used during the visit. The OPM-MEG visit will take place during the day when the infant is well-rested. Upon arrival, the infant will be fitted with a cotton bonnet that ties under the chin and has velcro that will attach to the inside of the 3D-printed MEG cap. This bonnet helps secure the MEG cap to the infant's head while providing some additional comfort and protection from the warm sensors. Once the bonnet has been secured, and sensors have been placed in the MEG cap, the MEG cap can be placed on the infant's head. The cap will be secured using a bike helmet strap with chin support to prevent cap movement. Infants and their caregivers will enter the magnetically shielded room (MSR-see below for details) and approximately 10 sensors will be placed over the frontal lobe and primary sensory and motor cortices. The MSR is then degaussed to minimize outside magnetic field interference and the sensors are calibrated from the console room outside of the MSR. The MSR is outfitted with a microphone and camera to ensure constant communication with participants. Additionally, a well-trained researcher will remain in the room to address the needs of the family throughout the procedure. The OPM session is estimated to take no more than 20 minutes of sensor recording. Infants will be placed on their backs between two nulling coils (hardware that greatly diminishes the impact of any ambient magnetic fields that remain after degaussing-see below for details) to allow for naturalistic and free movement of their limbs and trunk consistent with the protocol of the GMA while ensuring the least amount of magnetic interference from outside the infant's brain.

Magnetically Shielded Room: The MSR forms a magnetically shielded volume using high-permeability mu-metal, with the trademark, MuRoom® (FIG. 2A). The inner dimensions of the room are approximately 3 m×3 m×2.4 m (9⅞ ft×9⅞ ft×7¾ ft). The magnetically shielded room acts to stop interference from external magnetic fields.

MEG Cap: The infant-sized OPM-MEG cap was designed in SolidWorks and 3D printed using the Ultimaker S5 3D printer and prepared with Ultimaker Cura software. The cap is easily modifiable to accommodate the infant's head size and shape. The rigid outer shell of the MEG cap is made from polylactic acid and sensors are secured into the cap using holders printed from TPU to help dissipate sensor heat.

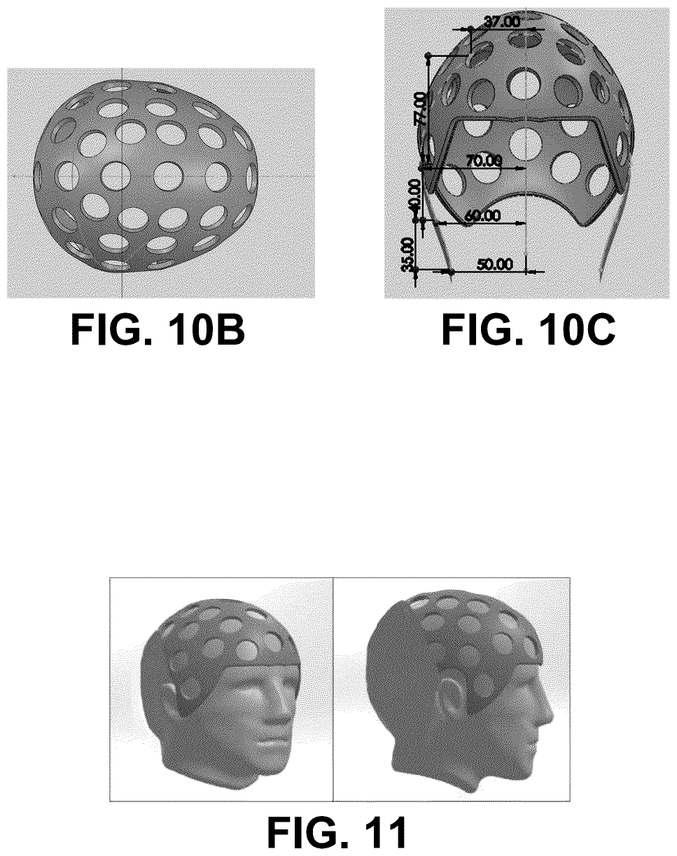

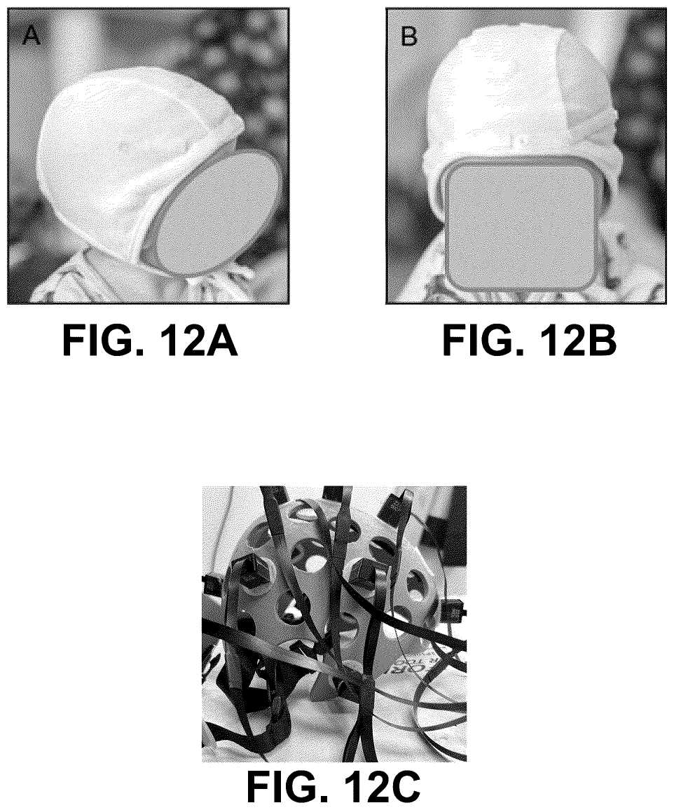

For the work to be carried out, modifications have been made to allow the real-time collection of the GMA while the infant is in the supine position (FIG. 11). Removing the back portion of the cap allows the infant to move their head using a full range of motion, while still allowing OPM recordings over the sensory and motor areas of interest.

QuSpin Generation Two Sensors: OPM sensors developed by and purchased from QuSpin are among the most sensitive magnetic field detectors available. Gen 2 sensors are approximately 12.4×16.6×24.4 mm and weigh 4 grams per sensor. The sensor contains three main components: the laser, high-pressurized rubidium (Rb) vapor, and a photodiode. The laser shines through the Rb vapor to elicit optical pumping resulting in highly polarized vapor. The sensors are field zeroed and when a magnetic field is detected the vapor knocks out of alignment, allowing measurement of the magnetic field. Sensors collect information simultaneously from two directions (diaxial), radial and tangential relative to the participant's head (Marhl 2022).

The OPMs are enclosed inside a 2×2×5 cm3 plastic housing. The principal components in the OPM include a very low power light-source (0.1 mW laser), a sealed glass cell containing (non-radioactive) alkali atoms, a silicon photodetector, and other off-the-shelf optical elements. OPMs function as simple passive detectors to measure magnetic field internally generated by the subject. There is no electromagnetic or laser radiation emitted by the OPM at any time. Additionally, there are no hazardous materials used within or during the construction of the OPMs.

The glass vapor-cell inside the QuSpin OPM is electrically heated to 150° C. Consequently, the sensor housing may get slightly warm (a few degrees centigrade above ambient, ˜41° C.) but poses no risk of injury, even upon direct physical contact. Even so, the possibility of any physical contact between the sensor and the subject is completely eliminated by the magnetometer helmet used to seat the sensors. Although it is unlikely, participants may still feel this heat. For participants who have sensitive skin, it is possible that the sensors could leave a red mark, which might persist for a short amount of time after scanning.

The sensor housing is constructed using high temperature, opaque plastic designed to withstand severe mechanical/thermal shock or electrical breakdown. The OPMs do not emit any harmful radiation (electromagnetic or particle). They are deemed completely safe during normal operation, as well as in the highly unlikely event of a catastrophic failure. Furthermore, the input power supply is +19 volts, which meets the OSHA definition of “low voltage.”

Nulling Coils: Two nulling coils (Cerca Magnetics, Nottingham, UK) will sit on both sides of the participant's body within the MSR to null any outside magnetic field that may produce interference and create noise within the data. The nulling coils are an essential component to collect high-quality OPM data, especially when motion is introduced (Holmes 2018). Two OPM reference sensors on each side (4 total) are secured to the nulling coils outside of the cap. These reference sensors actively monitor the external magnetic field and actively send feedback to the coils to null the field. Nulling coils will be particularly important in this population because of their age and the task they will be performing.

Temporal resolution: The data acquisition rate is 1200 Hz.

Spatial resolution: The spatial resolution is impacted by the distance from the sensor to the brain region recorded, as well as the number of sensors. With the current MEG cap design, we estimate that 10 sensors will provide adequate cortical coverage over the primary sensory and motor cortex.

Software: QuSpin software will be used to calibrate and monitor sensors during data collection. cMEG acquisition software (Cerca Magnetics, Nottingham, UK) will analyze incoming sensor recording data and give real-time feedback on external sources of noise that can be accounted for in post-processing.

### REFERENCES FOR EXAMPLE 2

1. CDC. Birth Prevalence of Cerebral Palsy|CDC. Centers for Disease Control and Prevention. 2020. (Accessed 18 Apr. 2023).
2. A report: the definition and classification of cerebral palsy April 2006. Dev Med Child Neurol 2007; 49:8-14. doi: 10.1111/j.1469-8749.2007. tb12610.x.
3. Novak I, Morgan C, Adde L, Blackman J, Boyd R N, Brunstrom-Hernandez J, et al. Early, Accurate Diagnosis and Early Intervention in Cerebral Palsy: Advances in Diagnosis and Treatment. JAMA Pediatr 2017; 171:897-907. doi: 10.1001/jamapediatrics.2017.1689.
4. Glass H C, Li Y, Gardner M, Barkovich A J, Novak I, McCulloch C E, et al. Early Identification of Cerebral Palsy Using Neonatal MRI and General Movements Assessment in a Cohort of High-Risk Term Neonates. Pediatr Neurol 2021; 118:20-5. doi: 10.1016/j.pediatrneurol.2021.02.003.
5. Kline J E, Sita Priyanka Illapani V, He L, Parikh N A. Automated brain morphometric biomarkers from MRI at term predict motor development in very preterm infants. Neurolmage Clin 2020; 28:102475. doi: 10.1016/j.nicl.2020.102475.
6. Duerden E G, Foong J, Chau V, Branson H, Poskitt K J, Grunau R E, et al. Tract-Based Spatial Statistics in Preterm-Born Neonates Predicts Cognitive and Motor Outcomes at 18 Months. Am J Neuroradiol 2015; 36:1565-71. doi: 10.3174/ajnr.A4312.
7. Kline J E, Yuan W, Harpster K, Altaye M, Parikh N A. Association between brain structural network efficiency at term-equivalent age and early development of cerebral palsy in very preterm infants. Neurolmage 2021; 245:118688. doi: 10.1016/j.neuroimage.2021.118688.
8. Boto E, Hill R M, Rea M, Holmes N, Seedat Z A, Leggett J, et al. Measuring functional connectivity with wearable MEG. Neurolmage 2021; 230:117815. doi: 10.1016/j.neuroimage.2021.117815.
9. Boto E, Shah V, Hill R M, Rhodes N, Osborne J, Doyle C, et al. Triaxial detection of the neuromagnetic field using optically-pumped magnetometry: feasibility and application in children. Neurolmage 2022; 252:119027. doi: 10.1016/j.neuroimage.2022.119027.
10. Brookes M J, Leggett J, Rea M, Hill R M, Holmes N, Boto E, et al. Magnetoencephalography with optically pumped magnetometers (OPM-MEG): the next generation of functional neuroimaging. Trends Neurosci 2022: S0166223622001023. doi: 10.1016/j.tins.2022.05.008.
11. Mcintyre S, Taitz D, Keogh J, Goldsmith S, Badawi N, Blair E. A systematic review of risk factors for cerebral palsy in children born at term in developed countries. Dev Med Child Neurol 2013; 55:499-508. doi: 10.1111/dmcn.12017.
12. Prechtl H F, Einspieler C, Cioni G, Bos A F, Ferrari F, Sontheimer D. An early marker for neurological deficits after perinatal brain lesions. Lancet Lond Engl 1997; 349:1361-3. doi: 10.1016/S0140-6736 (96) 10182-3.
13. Einspieler C, Bos A F, Libertus M E, Marschik P B. The General Movement Assessment Helps Us to Identify Preterm Infants at Risk for Cognitive Dysfunction. Front Psychol 2016; 7:406. doi: 10.3389/fpsyg.2016.00406.
14. Bcl T. Examination of the child with minor neurological dysfunction. Clin Dev Med 1979; 71:56-70.
15. Ceschin R, Lee V K, Schmithorst V, Panigrahy A. Regional vulnerability of longitudinal cortical association connectivity: Associated with structural network topology alterations in preterm children with cerebral palsy. Neurolmage Clin 2015; 9:322-37. doi: 10.1016/j.nicl.2015.08.021.
16. Coker-Bolt P, Barbour A, Moss H, Tillman J, Humphries E, Ward E, et al. Correlating early motor skills to white matter abnormalities in preterm infants using diffusion tensor imaging. J Pediatr Rehabil Med 2016; 9:185-93. doi: 10.3233/PRM-160380.
17. Dall′Orso S, Steinweg J, Allievi A G, Edwards A D, Burdet E, Arichi T. Somatotopic Mapping of the Developing Sensorimotor Cortex in the Preterm Human Brain. Cereb Cortex 2018; 28:2507-15. doi: 10.1093/cercor/bhy050.
18. De Bruine F T, Van Wezel-Meijler G, Leijser L M, Steggerda S J, Van Den Berg-Huysmans A A, Rijken M, et al. Tractography of white-matter tracts in very preterm infants: a 2-year follow-up study. Dev Med Child Neurol 2013; 55:427-33. doi: 10.1111/dmcn.12099.
19. De Wel O, Van Huffel S, Lavanga M, Jansen K, Dereymaeker A, Dudink J, et al. Relationship Between Early Functional and Structural Brain Developments and Brain Injury in Preterm Infants. The Cerebellum 2021; 20:556-68. doi: 10.1007/s12311-021-01232-z.
20. Boto E, Meyer S S, Shah V, Alem O, Knappe S, Kruger P, et al. A new generation of magnetoencephalography: Room temperature measurements using optically-pumped magnetometers. Neurolmage 2017; 149:404-14. doi: 10.1016/j.neuroimage.2017.01.034.
21. Hill R M, Boto E, Rea M, Holmes N, Leggett J, Coles L A, et al. Multi-channel whole-head OPM-MEG: Helmet design and a comparison with a conventional system. Neurolmage 2020; 219:116995. doi: 10.1016/j.neuroimage.2020.116995.
22. Hill R M, Boto E, Holmes N, Hartley C, Seedat Z A, Leggett J, et al. A tool for functional brain imaging with lifespan compliance. Nat Commun 2019; 10:4785. doi: 10.1038/s41467-019-12486-x.
23. Stavsky M, Mor O, Mastrolia S A, Greenbaum S, Than N G, Erez O. Cerebral Palsy-Trends in Epidemiology and Recent Development in Prenatal Mechanisms of Disease, Treatment, and Prevention. Front Pediatr 2017; 5.
24. Dimitrova R, Pietsch M, Ciarrusta J, Fitzgibbon S P, Williams L Z J, Christiaens D, et al. Preterm birth alters the development of cortical microstructure and morphology at term-equivalent age. Neuroimage 2021; 243:118488. doi: 10.1016/j.neuroimage.2021.118488.
25. Thompson D K, Loh W Y, Connelly A, Cheong J L Y, Spittle A J, Chen J, et al. Basal ganglia and thalamic tract connectivity in very preterm and full-term children; associations with 7-year neurodevelopment. Pediatr Res 2020; 87:48-56. doi: 10.1038/s41390-019-0546-x.
26. Thompson D K, Inder T E, Faggian N, Johnston L, Warfield S K, Anderson P J, et al. Characterization of the corpus callosum in very preterm and full-term infants utilizing MRI. Neurolmage 2011; 55:479-90. doi: 10.1016/j.neuroimage.2010.12.025.
27. Spann M N, Wisnowski J L, Ahtam B, Gao W, Huang H, Nebel M B, et al. The Art, Science, and Secrets of Scanning Young Children. Biol Psychiatry 2023; 93:858-60. doi: 10.1016/j.biopsych.2022.09.025.
28. Dubois J, Dehaene-Lambertz G, Kulikova S, Poupon C, Hüppi P S, Hertz-Pannier L. The early development of brain white matter: a review of imaging studies in fetuses, newborns and infants. Neuroscience 2014; 276:48-71. doi: 10.1016/j.neuroscience.2013.12.044.
29. Woodward K E, Carlson H L, Kuczynski A, Saunders J, Hodge J, Kirton A. Sensory-motor network functional connectivity in children with unilateral cerebral palsy secondary to perinatal stroke. Neurolmage Clin 2019; 21:101670. doi: 10.1016/j.nicl.2019.101670.
30. Papadelis C, Ahtam B, Nazarova M, Nimec D, Snyder B, Grant P E, et al. Cortical Somatosensory Reorganization in Children with Spastic Cerebral Palsy: A Multimodal Neuroimaging Study. Front Hum Neurosci 2014; 8.
31. Chandwani R, Kline J E, Harpster K, Tkach J, Parikh N A, Group TCINEPS (CINEPS). Early micro- and macrostructure of sensorimotor tracts and development of cerebral palsy in high risk infants. Hum Brain Mapp 2021; 42:4708-21. doi: 10.1002/hbm.25579.
32. Sylvester C M, Kaplan S, Myers M J, Gordon E M, Schwarzlose R F, Alexopoulos D, et al. Network-specific selectivity of functional connections in the neonatal brain. Cereb Cortex N Y NY 2022; 33:2200-14. doi: 10.1093/cercor/bhac202.
33. Kurz M J, Bergwell H, Spooner R, Baker S, Heinrichs-Graham E, Wilson T W. Motor beta cortical oscillations are related with the gait kinematics of youth with cerebral palsy. Ann Clin Transl Neurol 2020; 7:2421-32. doi: 10.1002/acn3.51246.
34. Démas J, Bourguignon M, Périvier M, De Tiège X, Dinomais M, Van Bogaert P. Mu rhythm: State of the art with special focus on cerebral palsy. Ann Phys Rehabil Med 2020; 63:439-46. doi: 10.1016/j.rehab.2019.06.007.
35. Papadelis C, Butler E E, Rubenstein M, Sun L, Zollei L, Nimec D, et al. Reorganization of the somatosensory cortex in hemiplegic cerebral palsy associated with impaired sensory tracts. Neurolmage Clin 2018; 17:198-212. doi: 10.1016/j.nicl.2017.10.021.
36. Pihko E, Nevalainen P, Vaalto S, Laaksonen K, Mäenpää H, Valanne L, et al. Reactivity of sensorimotor oscillations is altered in children with hemiplegic cerebral palsy: A magnetoencephalographic study. Hum Brain Mapp 2014; 35:4105-17. doi: 10.1002/hbm.22462.
37. Marhl U, Jodko-Władzińska A, Brühl R, Sander T, Jazbinšek V. Transforming and comparing data between standard SQUID and OPM-MEG systems. PloS One 2022; 17: e0262669. doi: 10.1371/journal.pone.0262669.
38. Holmes N, Leggett J, Boto E, Roberts G, Hill R M, Tierney T M, et al. A bi-planar coil system for nulling background magnetic fields in scalp mounted magnetoencephalography. Neurolmage 2018; 181:760-74. doi: 10.1016/j.neuroimage.2018.07.028.
39. Rempe M P, Ott L R, Picci G, Penhale S H, Christopher-Hayes N J, Lew B J, et al. Spontaneous cortical dynamics from the first years to the golden years. Proc Natl Acad Sci 2023; 120: e2212776120 doi: 10.1073/pnas.2212776120.
40. The Electroencephalogram During Normal Infancy and Childhood: I. Rhythmic Activities Present in the Neonate and Their Subsequent Development: The Pedagogical Seminary and Journal of Genetic Psychology: Vol 53, No 2. n.d (Accessed 3 Aug. 2023).
41. Stroganova T A, Orekhova E V, Posikera I N. EEG alpha rhythm in infants. Clin Neurophysiol 1999; 110:997-1012. doi: 10.1016/S1388-2457 (98) 00009-1.
42. Howell B R, Styner M A, Gao W, Yap P-T, Wang L, Baluyot K, et al. The UNC/UMN Baby Connectome Project (BCP): An overview of the study design and protocol development. Neurolmage 2019; 185:891-905. doi: 10.1016/j.neuroimage.2018.03.049.

It should be emphasized that the above-described embodiments of the present disclosure are merely possible examples of implementations set forth for a clear understanding of the principles of the disclosure. Many variations and modifications may be made to the above-described aspects. All such modifications and variations are intended to be included herein within the scope of this disclosure and protected by the following claims.

What is claimed:

1. A helmet system, comprising:
a helmet shell configured to fit the head of a human subject, the helmet shell weighing approximately 450 grams or less;
at least one sensor housing unit, configured to hold a magnetoencephalography field sensor; and
a plurality of openings disposed in the helmet shell, wherein at least two individual openings of the plurality of openings are configured to receive and securely fit the sensor housing unit.

2. The helmet system of claim 1, wherein the helmet shell weighs approximately 100 grams to approximately 350 grams.

3. The helmet system of claim 1, wherein the helmet shell has a thickness of approximately 1 mm to approximately 6 mm.

4. The helmet system of claim 1, wherein the helmet shell is comprised of polylactic acid, polyethylene terephthalate glycol, or a combination thereof.

5. The helmet system of claim 1, wherein the helmet shell is configured to fit the head of a human subject of approximately 2 months to approximately 24 months old.

6. The helmet system of claim 1, wherein the helmet shell is configured to fit the head of a human subject with an occipital frontal circumference of approximately 35 cm to approximately 55 cm.

7. The helmet system of claim 1, wherein the helmet shell is configured to fit the head of a human subject with an occipital frontal circumference of approximately 35 cm to 55 cm.

8. The helmet system of claim 1, wherein the helmet shell is configured to fit a head shape characterized by normocephaly, brachycephaly, plagiocephaly, scaphocephaly, trigonocephaly, asymmetrical ears, or any combination thereof.

9. The helmet system of claim 1, wherein the sensor housing unit is comprised of thermoplastic copolyester, thermoplastic polyurethane, or a combination thereof.

10. The helmet system of claim 1, wherein the magnetoencephalography field sensor is an optically pumped magnetometer.

11. The helmet system of claim 1, wherein the plurality of openings comprises between 2 to approximately 50 openings in the helmet shell.

12. The helmet system of claim 1, wherein the plurality of openings each individually have a shape characterized as circular, oval, or polygonal.

13. The helmet system of claim 12, wherein each shape individually has a longest diameter ranging from approximately 5 mm to approximately 25 mm.

14. The helmet system of claim 1, further comprising at least one inflatable air bladder attached to an inner surface of the helmet shell.

15. The helmet system of claim 14, wherein the inflatable air bladder, padding, or both is comprised of a high molecular weight polyethylene or a derivative thereof.

16. A kit, comprising:
the helmet system of claim 1; and
at least one magnetoencephalography field sensor.

17. The kit of claim 16, wherein the magnetoencephalography field sensor is an optically pumped magnetometer.

18. The kit of claim 16, further comprising a cap configured to fit the head of the human subject.
